(19) United States
(12) Patent Application Publication
(10) Pub. No.: US 2026/0273038 A1
(43) Pub. Date: Sep. 17, 2026

(54) COMPOSITIONS AND METHODS FOR PREVENTING, CONTROLLING AND DIAGNOSING MYCOBACTERIAL INFECTIONS

(71) Applicant: University of Saskatchewan, Saskatoon (CA)

(72) Inventors: Antonio Facciuolo, Saskatoon (CA); Philip Griebel, Saskatoon (CA); Volker Gerdts, Saskatoon (CA); Andrew Potter, Saskatoon (CA); Neil Rawlyk, Saskatoon (CA); Jeffrey Chen, Saskatoon (CA); Elodie Pastural, Saksatoon (CA); Manjeet Bains, Vancouver (CA); Michael Trimble, Vancouver (CA); Amy Lee, Vancouver (CA); Robert Hancock, Vancouver (CA)

(21) Appl. No.: 19/456,028

(22) Filed: Jan. 22, 2026

Related U.S. Application Data
(63) Continuation of application No. 17/996,688, filed on Apr. 19, 2021, now abandoned.
(60) Provisional application No. 63/012,668, filed on Apr. 20, 2020.

(51) Int. Cl. A61K 39/04; A61K 39/00; A61K 39/39; A61P 31/04; G01N 33/569

(52) U.S. Cl. CPC A61K 39/04; A61K 39/39; A61P 31/04; G01N 33/5695; A61K 2039/55566; G01N 2469/20

(57) ABSTRACT

Mycobacterial antigens, such as MAP and M. bovis antigens, are described. The antigens can be used in subunit compositions to elicit immune responses in order to prevent and/or control mycobacterial infections, as well as in diagnostics in order to detect mammals infected with mycobacteria.

### CROSS-REFERENCE TO RELATED APPLICATIONS

This application is a continuation of U.S. patent application Ser. No. 17/996,688, filed Oct. 20, 2022, which is a National Stage Entry under 35 U.S.C. § 371 of International Patent Application No. PCT/CA2021/050527, filed Apr. 19, 2021, which claims the benefit of priority of U.S. Provisional Patent Application No. 63/012,668, filed Apr. 20, 2020. The foregoing applications are incorporated by reference herein in their entireties.

### TECHNICAL FIELD

The present invention relates generally to bacterial pathogens. In particular, the invention pertains to compositions and methods for preventing, controlling and diagnosing mycobacterial infections, such as infections caused by Mycobacterium avium subspecies paratuberculosis (MAP) and Mycobacterium bovis (M. bovis).

### BACKGROUND

Pathogenic mycobacteria are etiological agents of human and veterinary diseases with significant mortality, morbidity and economic impact worldwide. To date, only one vaccine, bacillus of Calmette and Guérin (BCG), has been used to prevent tuberculosis (TB) infection in humans and economically important livestock species such as cattle. The BCG vaccine is a live-attenuated strain of Mycobacterium bovis (M. bovis) that was developed a century ago to protect against Mycobacterium tuberculosis (Mtb) in humans. Since then, regional variants of the BCG strain have been developed and continue to be widely implemented in tuberculosis (TB) control programs despite showing highly variable levels of protection among human populations (Vaudry W. Paediatric Child Health (2003) 8:141-144). In addition, BCG is known to frequently cause a local reaction at the vaccine injection site consistent with primary infection with an attenuated strain, such as small localized ulcer and possible regional lymphadenopathy.

Efficacious vaccines that afford a high level of protection against infection for mycobacterial pathogens affecting terrestrial and aquatic species have not been developed. The complex nature of chronic mycobacterial infections, in addition to the arsenal of both defined and undefined virulence factors used by mycobacteria to evade the host immune system, has provided a major hurdle in vaccine development.

M. bovis is the causative agent of bovine tuberculosis (bTB). bTB is a chronic infectious pulmonary disease that affects cattle and a broad range of mammalian species including humans, deer, llamas, pigs, domestic cats, wild carnivores and omnivores. Transmission of M. bovis is facilitated primarily by cough-aerosols, but infected hosts can contaminate the surrounding environment by excretion of the bacterium in urine, faeces, and pus. An effective vaccine against M. bovis in cattle is needed to prevent infection in economically important livestock species, and could also serve to reduce the risk of zoonotic infection.

Mycobacterium avium subspecies paratuberculosis (MAP) is the causative agent of Johne's disease, a chronic gastroenteritis of ruminants. MAP infections are endemic worldwide with high prevalence rates in dairy cattle (Corbett et al., J. Dairy Sci. (2018) 101:11218-11228), sheep and goat herds (Bauman et al., Can. Vet. J. (2016) 57:169-175). Johne's disease results in significant economic losses to the dairy and beef industry (Garcia et al., J. Dairy Sci. (2015) 98:5019-5039), primarily due to decreased milk production (Smith et al., J. Dairy Sci. (2009) 92:2653-2661; McAloon et al., J. Dairy Sci. (2016) 99:1449-1460) and slaughter value (Kudahl et al., J. Dairy Sci. (2009) 92:4340-4346; Roy et al., Can. Vet. J. (2017) 58:296-298). MAP-infected cattle can remain asymptomatic for years following infection (Whitlock et al., Vet. Clin. North Am. Food Animal Prac. (1996) 12:345-356). However, intermittent shedding of MAP bacteria in faeces (Crossley et al., Vet. Microbiol. (2005) 107:257-263) and milk (Stabel et al., J. Dairy Sci. (2014) 97:6296-6304) sustains transmission from cow to calf (Benedictus et al., Prev. Vet. Med. (2008) 83:215-227; Patterson et al., Prev. Vet. Med. (2019) In Press) and among calves (van Roermund et al., Vet. Microbiol. (2007) 122:270-279; Wolf et al., Vet. Res. (2015) 46:71).

Detection of MAP bacteria in the environment (Smith et al., (2011) Prev. Vet. Med. 102:1-9), drinking water (Chern et al., J. Water Health (2015) 13:131-139) and retail milk (Gerrard et al., Food Microbiol. (2018) 74:57-63) has led to food safety concerns, specifically transmission of infection to humans. A growing body of evidence has implicated MAP in human Crohn's and other autoimmune diseases (Sechi et al., Front Immunol. (2015) 6:96; Waddell et al., Epidemiol. Infect. (2015) 143:3135-3157; Timms et al., PLOS One (2016) 11: e0148731; Kuenstner et al., Front Public Health (2017) 5:208). Higher bioloads of MAP in animals may increase the risk of exposure to the human population.

Efforts to combat MAP-related diseases (i.e. Johne's disease) are dependent on specific and sensitive detection of infected animals, as well as development of vaccines that can control or prevent infections. Diagnosis and control, however, are problematic due in part to the long incubation period of the disease during which infected animals show no clinical signs so that infection is difficult to detect. Additionally, MAP is able to survive and persist in the environment for long periods of time. Commercially available diagnostic tests for MAP-infected animals, such as serological based enzyme-linked immunosorbent assays (ELISAs), although displaying high specificity, often fail to detect most MAP-infected animals due to low sensitivity (less than 40%; Clark et al., J. Dairy Sci. (2008) 91:2620-2627). Furthermore, these tests do not identify infected younger animals in the early stages of infection (less than 1-2 years old; Mortier et al., J. Dairy Sci. (2014) 97:5558-5565). The tests are most sensitive (70-80%) in older animals in the later stages of infection (Jenvey et al., Vet. Immunol. Immunopathol. (2018) 202:93-101) and in those animals shedding high numbers of MAP bacteria (Clark et al., J. Dairy Sci. (2008) 91:2620-2627). As these serological-based tests are dependent on the antigen composition, a better understanding of MAP antigens can aid in the development of more sensitive diagnostic tests. Similarly, current diagnostic tests for M. bovis (e.g. BOVIGAM®, tuberculin skin test) are unreliable due to low specificity and sensitivity, particularly in younger animals.

Vaccination has not been widely used in cattle for MAP or M. bovis infection, in part due to the need to readily distinguish vaccinated from infected animals. The ability to readily distinguish vaccinated from infected animals is important for mycobacterial diseases, because current government policies dictate slaughter as a control method for cattle that test positive in a bovine tuberculosis test. Companion diagnostics are therefore necessary to discriminate between infected and vaccinated animals.

Current MAP vaccines are based on inactivated whole-cells administered parenterally. These vaccines do not prevent infection but can reduce fecal shedding and delay onset of clinical disease (Barkema et al., Transbound. Emerg. Dis. (2018) 1:125-148). Traditional approaches to vaccine design for mycobacterial species have proven largely unsuccessful. Vaccine development is difficult, especially in slow-growing Mycobacterium species, due in part to the inefficient and often ineffective identification of protective antigens using traditional methods.

It is apparent that the identification of antigens for use in diagnostics for detecting mycobacterial infections, such as MAP and M. bovis infections, and in vaccine compositions for controlling and/or preventing mycobacterial infections, is needed.

### SUMMARY OF THE INVENTION

The identification and selection of mycobacterial antigens, including without limitation, MAP and M. bovis antigens, for use in vaccine compositions, such as subunit vaccine compositions, and as diagnostics, are described herein. MAP and M. bovis share more than 3000 genes encoding homologous proteins (Li et al. Proc. Natl/Acad. Sci. USA. (2005) 102 (35): 12344-12349). Moreover, M. bovis and MAP cause disease in the same host (e.g. ruminants). Hence, the identification of antigens from these mycobacterial species provides an opportunity to identify antigens that provide cross-protection mediated by protective T and/or B cell responses against both pathogens, as well as against other mycobacterial species sharing homologous or orthologous proteins, such as M. tuberculosis (Mtb).

The inventors herein have identified several unique MAP and M. bovis proteins using reverse vaccinology that provides a relatively unbiased genomic strategy for selecting protein antigens (and DNA encoding these antigens) for use in vaccine and diagnostics development. Reverse vaccinology, also termed vaccinomics, uses in silico processes to define a potential set of antigens from the genome sequence of an organism based on various information including the localization of the antigens within cells. A greater understanding of the underlying biology and virulence mechanisms of mycobacteria, by determining which antigens are expressed in vivo, guides the selection of proteins for vaccine and diagnostic design.

Using this approach, several antigenic mycobacterial protein candidates were identified and genes coding for candidate proteins were synthesized, expressed, and the corresponding recombinant proteins purified. Further analyses indicated that several of the proteins were immunogenic.

Accordingly, the present invention provides mycobacterial compositions for the prevention and/or control of mycobacterial infection, such as, but not limited to, MAP, M. bovis and/or Mtb infection. Subunit vaccine compositions have the advantage of allowing recombinant antigen production to be performed in host cells, such as E. coli, which provides a safe, rapid and inexpensive alternative to vaccines that require growth, attenuation and inactivation of mycobacteria. Subunit compositions, including immunogens and mixtures of immunogens derived from mycobacteria, such as MAP and M. bovis isolates, can also be used to diagnose mycobacterial infection. The present invention thus provides a commercially useful method of controlling, preventing and/or diagnosing mycobacterial infection in mammals, as well as for differentiating infected animals from vaccinated animals (DIVA).

Accordingly, in one embodiment, an immunogenic, subunit composition is provided. The composition comprises a pharmaceutically acceptable excipient and at least one isolated, mycobacterial antigen selected from (a) a MAP antigen, or an ortholog thereof, wherein the MAP antigen or ortholog is from Tables 1, 2, 3, 4, or 5; (b) a M. bovis antigen from Tables 2 or 5; an immunogenic fragment of (a) or (b); an immunogenic variant of (a) or (b); or the corresponding antigen from another mycobacterial strain or isolate, with the proviso that the selected mycobacterial antigen is not MAP2785c or MAP1981c.

In additional embodiments, the MAP antigen is selected from one or more of the MAP antigens from Tables 3 or 4, an immunogenic fragment thereof, or an immunogenic fragment or variant thereof. In certain embodiments, the MAP antigen or ortholog comprises an amino acid sequence with at least 99% sequence identity to a MAP antigen or ortholog from Tables 1, 2, 3, 4, or 5.

In further embodiments, the M. bovis antigen is selected from one or more of the M. bovis antigens from Table 5, an immunogenic fragment thereof, or an immunogenic fragment or variant thereof. In certain embodiments, the M. bovis antigen comprises an amino acid sequence with at least 99% sequence identity to a M. bovis antigen from Tables 2 or 5.

In additional embodiments, the he mycobacterial antigen in the immunogenic composition comprises a deletion of all or part of a transmembrane binding domain or a native signal sequence, if present.

In other embodiments, the immunogenic composition comprises two or more isolated mycobacterial antigens selected from MAP antigens or orthologs and/or M. bovis antigens or orthologs, from Tables 1, 2, 3, 4, 5, or 6, or an immunogenic fragment or variant thereof. In certain embodiments, the two or more antigens are provided as a fusion protein.

In additional embodiments, the immunogenic composition comprises an immunological adjuvant, such as, but not limited to an immunological adjuvant that comprises an oil-in-water emulsion.

In further embodiments, the immunological adjuvant comprises (a) a polyphosphazene; (b) a poly(I:C) or a CpG oligonucleotide; and (c) a host defense peptide. In certain embodiments, the immunological adjuvant is in the form of a microparticle.

In additional embodiments, a method of preventing and/or controlling a mycobacterial infection in a mammalian subject is provided. The mycobacterial infection, can be, but is not limited to a MAP, M. bovis, or Mtb infection. The method comprises administering a therapeutic amount of any one of the compositions described herein to the subject.

In further embodiments, the subject is a bovine or ovine subject. In certain embodiments, the MAP infection comprises Johne's disease. In other embodiments, the M. bovis or Mtb infection comprises tuberculosis.

In additional embodiments, the subject is a human subject. In certain embodiments, the MAP infection comprises a gastrointestinal disorder. In further embodiments, the M. bovis or Mtb infection comprises tuberculosis.

In further embodiments, a method for reducing colonization of a Mycobacterium and/or reducing shedding in a mammalian subject is provided. In certain embodiments, the Mycobacterium is selected from, but not limited to, a MAP, M. bovis, or Mtb. The method comprises administering a therapeutically effective amount of any one of the compositions described herein to the subject.

In additional embodiments, a method of detecting mycobacterial antibodies in a biological sample is provided. The method comprises (a) providing a biological sample; (b) reacting the biological sample with one or more mycobacterial antigens from Tables 1, 2, 3, 4 or 5, an immunogenic fragment or variant thereof, or the corresponding antigen from another mycobacterial strain or isolate, under conditions which allow mycobacterial antibodies, when present in the biological sample, to bind to the one or more antigens to form an antibody/antigen complex; and (c) detecting the presence or absence of the complex, thereby detecting the presence or absence of mycobacterial antibodies in the sample.

In further embodiments, an immunodiagnostic test kit for detecting mycobacterial infection is provided. The test kit comprises one or more mycobacterial antigens from Tables 1, 2, 3, 4 or 5, an immunogenic fragment or variant thereof, or the corresponding antigen from another mycobacterial strain or isolate, and instructions for conducting the immunodiagnostic test.

These and other embodiments of the invention will readily occur to those of skill in the art in view of the disclosure herein.

### BRIEF DESCRIPTION OF THE FIGURES

FIG. 1 shows the weight change, in grams, between the day of M. bovis challenge and the day of euthanasia of mice immunized with a placebo vaccine, BCG, or a pool of five antigens, during mouse Trials 2, 3, and 4, described in the examples. Dots represent weight changes for individual mice. The bars represent the median weight change of each treatment group.

FIGS. 2A and 2B show the colony forming units (CFU) per gram of tissue collected from mice vaccinated with a placebo vaccine, BCG, or a pool of five antigens, in mouse Trials 1 and 2, as described in the examples. FIG. 2A shows lung results and FIG. 2B shows spleen results. Dots represent CFU recovery for individual mice. The bars represent the median CFU count of each treatment group.

FIGS. 3A and 3B show the linear regression analysis conducted on the weight gain and the CFU count of the organs of each individual mouse in Trial 2. Each animal is identified by a dot with two coordinates, CFU, and weight gain. FIG. 3A shows spleen results and FIG. 3B shows lung results.

FIGS. 4A-4D show the interferon gamma (IFNγ) titres obtained by stimulation with the individual proteins that composed the three antigen pools showing some protection as assessed by CFU counts and weight gain in the four mouse trials. The splenocytes of calves numbered as 74, 76, 77, 78, 79, and 81 which were challenged with M. bovis, were stimulated with bPPD (FIG. 4A; positive control), one of the 15 proteins as described herein (FIGS. 4B and 4C), or phosphate-buffered saline [PBS] (FIG. 4D; negative control).

### DETAILED DESCRIPTION OF THE INVENTION

The present invention will employ, unless otherwise indicated, conventional methods of microbiology, virology, chemistry, biochemistry, recombinant DNA techniques and immunology, within the skill of the art. Such techniques are explained fully in the literature. See, e.g., Fundamental Virology, Current Edition, vol. I & II (B. N. Fields and D. M. Knipe, eds.); Methods in Microbiology series Volumes 1-47 (Various editors, Academic Press Elsevier); Handbook of Experimental Immunology, Vols. I-IV (D. M. Weir and C. C. Blackwell eds., Blackwell Scientific Publications); T. E. Creighton, Proteins: Structures and Molecular Properties (W.H. Freeman and Company); A. L. Lehninger, Biochemistry (Worth Publishers, Inc., current edition); Sambrook, et al., Molecular Cloning: A Laboratory Manual (current edition); Methods In Enzymology (S. Colowick and N. Kaplan eds., Academic Press, Inc.).

All publications, patents and patent applications cited herein, whether supra or infra, are hereby incorporated by reference in their entireties.

The following amino acid abbreviations are used throughout the text:

| | |
|---|---|
| Alanine: Ala (A) | Arginine: Arg (R) |
| Asparagine: Asn (N) | Aspartic acid: Asp (D) |
| Cysteine: Cys (C) | Glutamine: Gln (Q) |
| Glutamic acid: Glu (E) | Glycine: Gly (G) |
| Histidine: His (H) | Isoleucine: Ile (I) |
| Leucine: Leu (L) | Lysine: Lys (K) |
| Methionine: Met (M) | Phenylalanine: Phe (F) |
| Proline: Pro (P) | Serine: Ser (S) |
| Threonine: Thr (T) | Tryptophan: Trp (W) |
| Tyrosine: Tyr (Y) | Valine: Val (V) |

### 1. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an antigen” includes a mixture of two or more such antigens, and the like.

By “Mycobacterium” is meant a bacterium of any species, subspecies, strain or isolate of the bacterial genus Mycobacterium. The term intends any member of the Mycobacterium tuberculosis complex (MTC), non-tuberculous Mycobacterium (NTM), and M. leprae. The MTC is a genetically homogeneous group characterized by approximately 99.9% similarity at the nucleotide level and identical 16S rRNA sequences. They however differ widely in terms of their host tropisms, phenotypes, and pathogenicity. The MTC includes, without limitation, M. bovis; M. tuberculosis; M. africanum; M. microti; M. canettii; M. caprae; M. pinnipedii; M. suricattae; M. mungi; M. dassie; and Mycobacterium oryx, among other species.

NTMs include all mycobacteria except the MTCs and M. leprae. Numerous NTM species have been identified, including, without limitation, M. avium, such as but not limited to, Mycobacterium avium subspecies paratuberculosis (MAP); M. intracellulare; M. kansasii; M. abscessus; M. chelonae; M. fortuitum; M. marinum; M. simiae; M. ulcerans; M. xenopi, among others.

The term “MAP” intends any strain or isolate of Mycobacterium avium subspecies paratuberculosis which is capable of causing infection and/or disease as described herein. For a review of MAP pathogenic microbes, see, e.g., Stevenson, K., Vet. Res. (2015) 46:64.

The term “M. bovis” intends any strain or isolate of the M. bovis species which is capable of causing infection and/or disease as described herein. For a review of M. bovis, see, e.g., Olea-Popelka et al. The Lancet Infectious Diseases (2017) 17: e21-e25; El-Sayed et al, Zoonoses and Public Health (2016); 63:251-264.

The terms “mycobacterial disease” and “mycobacterial disorder” are used interchangeably herein and refer to any disorder caused in a host organism by a Mycobacterium, such as, but not limited to, by MAP, M. bovis, or M. tuberculosis (Mtb).

The terms “MAP disease” and “MAP disorder” are used interchangeably herein and refer to any disorder caused in whole or in part by a MAP bacterium. As explained herein, MAP causes a chronic, progressive granulomatous enteritis known as Johne's disease, or paratuberculosis, in ruminants and other mammals. Bacteria can be transmitted to humans by MAP-infected animals that shed the bacterium into faeces and milk. MAP infection in humans can contribute to the etiology of inflammatory bowel disease (IBD), Crohn's disease, and other chronic gastrointestinal disorders. Infection in ruminants often remains asymptomatic for a number of years. Although symptoms of the disease are not observed, the ability to spread the pathogen through shedding in the faeces and milk remains. Thus, the term intends both clinical and subclinical disease.

The terms “M. bovis disease” and “M. bovis disorder” are used interchangeably herein and refer to a disease or disorder caused in whole or in part by a M. bovis bacterium. M. bovis is a slow-growing (16- to 20-hour generation time) aerobic bacterium and is the causative agent of tuberculosis and resultant pulmonary disorders in cattle (known as bovine TB). M. bovis can jump the species barrier and cause tuberculosis-like infection in humans and other mammals. It has the broadest host range of any member of the MTC. Bovine tuberculosis is a chronic and often deadly infectious disease that affects a broad range of mammalian hosts, including humans; cattle; deer; llamas; pigs; domestic cats; wild carnivores (e.g., foxes and coyotes); omnivores (e.g., common brushtail possum, mustelids and rodents); equids; and sheep. The disease can be transmitted in several ways, for example, through exhaled air, sputum, urine, faeces, and pus. Thus, the disease can be transmitted from one animal to another, or from an infected mammal to humans, such as through direct contact, contact with the excreta of an infected animal, or inhalation of aerosols, depending on the species involved. Transmission of M. bovis to humans generally occurs after close contact with infected animals, such as by occupational exposure, generally through inhalation of aerosols exhaled by infected mammals, including humans, or by consumption of unpasteurised contaminated dairy products.

The terms “Mtb disease” and “Mtb disorder” are used interchangeably herein and refer to a disease or disorder caused in whole or in part by an M. tuberculosis bacterium. Mtb is typically spread through the air when a person with tuberculosis infection in the lungs or throat coughs, speaks or sings, and people nearby breathe in the bacteria. Tuberculosis typically affects the lungs, and can also affect other parts of the body, including the kidney, spine, and brain. Tuberculosis infection can be symptomatic or asymptomatic. For example, people with latent infection harbor Mtb bacteria in their bodies but are not sick and cannot spread the bacteria to others. Many people with latent disease never develop active tuberculosis. Individuals with active disease, however, are sick and can transmit the bacteria to others.

The term “infection” refers to the presence of mycobacteria in a host organism. An infected organism can show symptoms of a mycobacterial disease or can be asymptomatic.

As used herein, the term “colonization” refers to the presence of mycobacteria in a particular organ targeted by the mycobacterial species. An animal or human colonized with a particular Mycobacterium does not necessarily display symptoms of infection. In the case of MAP, colonization typically refers to the presence of MAP in the intestinal tract of a mammal, such as, but not limited to, a ruminant or human. In the case of M. bovis, colonization typically refers to the presence of M. bovis in the lungs and lung-associated lymph nodes (e.g. tracheobronchial) of a mammal, such as, but not limited to, a ruminant or human. Mtb colonization can occur in the lungs, throat, oropharynx, kidney, spine, and brain, as well as lymph nodes (e.g., in the cervical lymph nodes) of a human or non-human primate, as well as in other mammals.

As used herein, the term “shedding” refers to the presence of bacteria in the excreta and/or secretions from an infected mammal, such as, but not limited to, mucous, sputum, cough, tears, milk, nasal secretions, urine, faeces, pus, perspiration, and the like. MAP shedding generally refers to the presence of MAP in the milk or faeces from an infected mammal. M. bovis shedding typically refers to the presence of the Mycobacterium in the cough, milk, nasal secretions or faeces from an infected mammal. Mtb shedding typically refers to the presence of Mycobacterium in the cough, nasal secretions or faeces from an infected animal, including an infected human.

The term “derived from” is used herein to identify the original source of a molecule but is not meant to limit the method by which the molecule is made which can be, for example, by chemical synthesis or recombinant means.

A “MAP molecule” is a molecule derived from a MAP bacterium, including, without limitation, polypeptide, protein, glycoprotein, antigen, polynucleotide, oligonucleotide, lipid, glycolipid, and nucleic acid molecules from any of the various MAP strains or isolates. The molecule need not be physically derived from the particular bacterium in question, but may be synthetically or recombinantly produced. Nucleic acid and polypeptide sequences from a number of MAP isolates are known and/or described herein. Representative MAP proteins, and polynucleotides encoding the proteins, for use in controlling and/or preventing infection, or in diagnostics, are presented in Tables 1, 3 and 4. A MAP molecule, such as an antigen, as defined herein, is not limited to those described in the tables, as various isolates are known and variations in sequences may occur between them. Additional representative sequences found in isolates from various mammals are listed in the National Center for Biotechnology Information (NCBI) database. See, also, Stevenson, K., Vet. Res. (2015) 46:64. Thus, a “MAP” molecule as defined herein intends a molecule from a MAP isolate or strain that corresponds to the particular MAP source molecule.

An “M. bovis molecule” is a molecule derived from an M. bovis bacterium, including, without limitation, polypeptide, protein, glycoprotein, antigen, polynucleotide, oligonucleotide, lipid, glycolipid, and nucleic acid molecules from any of the various M. bovis strains or isolates. The molecule need not be physically derived from the particular bacterium in question, but may be synthetically or recombinantly produced. Nucleic acid and polypeptide sequences from a number of M. bovis isolates are known and/or described herein. Representative M. bovis proteins, and polynucleotides encoding the proteins, for use in controlling and/or preventing infection, or in diagnostics, are presented in Table 2. An M. bovis molecule, such as an antigen, as defined herein, is not limited to those described in the tables, as various isolates are known and variations in sequences may occur between them. Additional representative sequences found in isolates from various mammals are listed in the National Center for Biotechnology Information (NCBI) database. See, also, Garnier et al. Proc. Natl. Acad. Sci. USA (2003) 100:7877-7882; mcobrowser.epf1.ch under M. bovis AF2122/97, a commonly studied strain of M. bovis. Thus, an “M. bovis” molecule as defined herein intends a molecule from an M. bovis isolate or strain that corresponds to the particular M. bovis source molecule.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation and the like. Furthermore, for purposes of the present invention, a “polypeptide” refers to a protein which includes modifications, such as deletions, additions and substitutions, to the native sequence, so long as the protein maintains the desired activity. These modifications may be deliberate, as through site-directed mutagenesis, or may be accidental, such as through mutations of hosts which produce the proteins, or through errors due to PCR amplification.

The term “peptide” as used herein refers to a fragment of a polypeptide. Thus, a peptide can include a C-terminal deletion, an N-terminal deletion and/or an internal deletion of the native polypeptide, so long as the entire protein sequence is not present. A peptide will generally include at least about 3-10 contiguous amino acid residues of the full-length molecule, and can include at least about 15-25 contiguous amino acid residues of the full-length molecule, or at least about 20-50 or more contiguous amino acid residues of the full-length molecule, or any integer between 3 amino acids and the number of amino acids in the full-length sequence, provided that the peptide in question retains the ability to elicit the desired immunological response.

As used herein, “immunization” or “immunize” refers to administration of a mycobacterial composition, such as, but not limited to, MAP or M. bovis, in an amount effective to stimulate the immune system of the animal to which the composition is administered, in order to elicit an immunological response against one or more of the antigens present in the composition.

By “immunogenic” protein, polypeptide or peptide is meant a molecule which includes one or more epitopes and thus can modulate an immune response. Such peptides can be identified using any number of epitope mapping techniques, well known in the art. See, e.g., Epitope Mapping Protocols in Methods in Molecular Biology (2018) (Johan Rockberg and Johan Nilvebrant, Eds.) Springer, New York. For example, linear epitopes may be determined by for example, software programs (See., e.g., Saha et al., Structure, Function, and Bioinformatics (2006) 65:40-48); or by concurrently synthesizing large numbers of peptides on solid supports, the peptides corresponding to portions of the protein molecule, and reacting the peptides with antibodies while the peptides are still attached to the supports. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Antigenic regions of proteins can also be identified using standard antigenicity and hydropathy plots, such as those calculated using, e.g., the Omiga software program available from the Oxford Molecular Group. This computer program employs the Hopp/Woods method, Hopp et al., Proc. Natl. Acad. Sci USA (1981) 78:3824-3828 for determining antigenicity profiles, and the Kyte-Doolittle technique, Kyte et al., J. Mol. Biol. (1982) 157:105-132 for hydropathy plots.

Immunogenic molecules, for purposes of the present invention, will usually be at least about 5 amino acids in length, such as at least about 10 to about 15 or more amino acids in length. There is no critical upper limit to the length of the molecule, which can comprise the full-length of the protein sequence, or even a fusion protein comprising two or more epitopes, proteins, antigens, etc.

As used herein, the term “epitope” generally refers to the site on an antigen which is recognized by a T- or B-cell receptor and/or an antibody. Several different epitopes may be carried by a single antigenic molecule. The term “epitope” also includes modified sequences of amino acids which stimulate responses against the whole organism. The epitope can be generated from knowledge of the amino acid and corresponding DNA sequences of the polypeptide, as well as from the nature of particular amino acids (e.g., size, charge, etc.) and the codon dictionary, without undue experimentation. See, e.g., Ivan Roitt, Essential Immunology; Janis Kuby, Immunology.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytotoxic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity, of nonspecific, effector cells against cells displaying peptide antigens in association with MHC molecules on their surface. A “cellular immune response” also refers to the production of cytokines, chemokines and other such molecules produced by activated T-cells, including those derived from CD4+ and CD8+ T-cells, and/or other white blood cells.

Thus, an immunological response as used herein may be one that stimulates the production of antibodies. The antigen of interest may also elicit production of CTLs. Hence, an immunological response may include one or more of the following effects: the production of antibodies by B-cells; and/or the activation of suppressor T-cells and/or memory/effector T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art, such as described in the Examples herein.

The innate immune system of mammals also recognizes and responds to molecular features of pathogenic organisms via activation of Toll-like receptors and similar pattern-recognition receptor molecules on immune cells. Upon activation of the innate immune system, various non-adaptive immune response cells are activated to, e.g., produce various cytokines, lymphokines and chemokines. Cells activated by an innate immune response include immature and mature dendritic cells of the monocyte and plasmacytoid lineage (MDC, PDC), as well as gamma/delta and alpha/beta T cell receptor cells, B cells and Natural Killer (NK) cells and other innate lymphoid cells. Thus, the present invention also contemplates an immune response wherein the immune response involves both an innate and adaptive response.

An “immunogenic composition” is a composition that comprises an immunogenic molecule where administration of the composition to a subject results in the development in the subject of a humoral and/or a cellular immune response to the molecule of interest. Additionally, an immunogenic composition includes compositions used in diagnostic applications.

An “antigen” refers to a molecule, such as a protein, polypeptide, or fragment thereof, containing one or more epitopes (either linear, conformational or both) that will stimulate a host's immune system to make a humoral and/or cellular antigen-specific response. The term is used interchangeably with the term “immunogen.” Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide which expresses an antigen or antigenic determinant in vivo, such as in DNA immunization applications, is also included in the definition of antigen herein.

As used herein, “vaccine” refers to a composition that serves to stimulate an immune response to a mycobacterial antigen, such as a MAP or M. bovis antigen, e.g., through use of an antigen from Tables 1, 2 and 3 herein. The immune response need not provide complete protection and/or control against mycobacterial infection, such as a MAP, M. bovis, or Mtb infection, or against colonization and shedding of mycobacteria, or transmissibility by mycobacteria. Even partial protection against colonization and shedding of mycobacteria, and/or reduction in chronic infections or transmissibility by mycobacteria, will find use herein. In some cases, a vaccine will include an immunological adjuvant in order to enhance the immune response. The term “adjuvant” refers to an agent which acts in a nonspecific manner to increase an immune response to a particular antigen or combination of antigens, thus reducing the quantity of antigen necessary in any given vaccine, and/or the frequency of injection necessary in order to generate an adequate immune response to the antigen of interest. Such adjuvants are described further below.

By “subunit composition,” such as a subunit vaccine, is meant a composition that includes one or more selected antigens but not all antigens, derived from or homologous to, an antigen from a pathogen of interest. Such a composition is substantially free of intact pathogen cells or pathogenic particles, or the lysate of such cells or particles. Thus, a “subunit composition” can be prepared from at least partially purified (preferably substantially purified) immunogenic molecules from the pathogen, or analogs thereof. The method of obtaining an antigen included in the subunit composition can thus include standard purification techniques, recombinant production, or synthetic production.

“Substantially purified” generally refers to isolation of a substance such that the substance comprises the majority percent of the sample in which it resides. Typically in a sample, a substantially purified component comprises at least 50%, preferably at least 80%-85%, more preferably at least 90-95%, such as at least 96%, 97%, 98%, 99%, or more of the sample. Techniques for purifying molecules of interest are well-known in the art and include, for example, ion-exchange chromatography, affinity chromatography and sedimentation according to density.

By “isolated” is meant that the indicated molecule is separate and discrete from the whole organism with which the molecule is found in nature or is present in the substantial absence of other biological macromolecules of the same type.

An “antibody” intends a molecule that “recognizes,” i.e., specifically binds to an epitope of interest present in an antigen. By “specifically binds” is meant that the antibody interacts with the epitope in a “lock and key” type of interaction to form a complex between the antigen and antibody, as opposed to non-specific binding that might occur between the antibody and, for instance, components in a mixture that includes the test substance with which the antibody is reacted. The term “antibody” as used herein includes antibodies obtained from both polyclonal and monoclonal preparations, as well as, the following: hybrid (chimeric) antibody molecules; F(ab′)₂ and F(ab) fragments; Fv molecules (non-covalent heterodimers; single-chain Fv molecules (sFv); dimeric and trimeric antibody fragment constructs; minibodies; humanized antibody molecules; and, any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)₂, Fv, and other fragments, as well as chimeric and humanized homogeneous antibody populations, that exhibit immunological binding properties of the parent monoclonal antibody molecule.

“Homology” refers to the percent identity between two polynucleotide or two polypeptide moieties. Two nucleic acid, or two polypeptide sequences are “substantially homologous” to each other when the sequences exhibit at least 75% to 99% or more sequence identity, such as at least about 80, 85, 90, 91, 92, 93, 94, 95, 96, 97, 98, 99, 99.5 or more percent sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis. See, e.g., molbiol-tools.ca/alignments for a list of computer programs to determine similarity between two or more amino acid or nucleotide sequences. These programs are readily utilized with the default parameters recommended by the manufacturer. For example, percent identity of a particular nucleotide sequence to a reference sequence can be determined using the homology Smith-Waterman algorithm with a default scoring table and a gap penalty of six nucleotide positions.

Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments.

DNA sequences that are substantially homologous can be identified in a Southern hybridization experiment under, for example, stringent conditions, as defined for that particular system. Defining appropriate hybridization conditions is within the skill of the art. See, e.g., Sambrook et al., Molecular Cloning, a laboratory manual, Cold Spring Harbor Laboratories, New York.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oregon, as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

“Recombinant” as used herein to describe a nucleic acid molecule means a polynucleotide of genomic, cDNA, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

“Recombinant host cells,” “host cells,” “cells,” “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, procaryotic or eucaryotic mRNA, genomic DNA sequences from viral or procaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

The term “transform” is used to refer to the uptake of foreign DNA by a cell. A cell has been “transformed” when exogenous DNA has been introduced inside the cell membrane. A number of transformation techniques are generally known in the art. See, e.g., Sambrook et al., Molecular Cloning, a laboratory manual, Cold Spring Harbor Laboratories, New York; Davis et al. Basic Methods in Molecular Biology, Elsevier. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses. When used for immunization, such gene delivery expression vectors may be referred to as vaccines or vaccine vectors.

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, secretions from the respiratory, intestinal, and genitourinary tracts, tears, saliva, milk, sputum, mucous, blood cells, organs, biopsies and also samples of in vitro cell culture constituents including but not limited to conditioned media resulting from the growth of cells and tissues in culture medium, e.g., recombinant cells, and cell components.

As used herein, the terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof which is capable of exhibiting fluorescence in the detectable range. Particular examples of labels which may be used under the invention include fluorescein, rhodamine, dansyl, umbelliferone, Texas red, luminol, NADPH and α-β-galactosidase.

As used herein, “preventing” infection refers to, without limitation, the prevention of infection or reinfection of a subject, such as through the administration of an immunogenic composition, e.g., a subunit vaccine composition that includes one or more antigens of interest, or the administration of an antibody composition to provide passive immunity. The term “preventing” also encompasses situations where the severity and/or length of active infection is lessened by an administered immunogenic composition.

The terms “controlling” infection and “treating” infection are used interchangeably herein and refer to, without limitation, the reduction or elimination of symptoms from an infected individual, as well as the reduction of the amount of bacteria present in a treated subject, or the amount of bacteria shed (e.g., secreted or excreted) by a treated subject. Treatment may be effected prophylactically (prior to infection) or therapeutically (following infection).

The prevention and/or treatment of a mycobacterial infection can include, for example, the prevention or reduction of colonization of mycobacteria in a treated subject, as well as the prevention or reduction in the number of mycobacteria shed from a treated subject, or the reduction of the time period of shedding by an animal. The location of colonization and the mode of shedding from an infected subject will vary, depending on the particular mycobacterial infection. For example, MAP typically colonizes the intestines, as well as distant organs, such as the liver and lymph nodes (e.g., ileum and the mesenteric lymph nodes), and bacteria are typically shed by an infected animal in milk and faeces. M. bovis, on the other hand, typically colonizes lung or lung-associated lymph nodes, and sheds bacteria through coughs and mucosal secretions.

As used herein, “therapeutic amount”, “effective amount” and “amount effective to” refer to an amount of vaccine effective to elicit an immune response against a selected mycobacterial species, such as, but not limited to, MAP or M. bovis antigen(s) present in a composition, thereby reducing or preventing MAP or M. bovis infection, disease, and/or colonization of a mammal such as a ruminant; and/or reducing the number of animals shedding mycobacteria, and/or reducing the number of mycobacteria shed by an animal; and/or, reducing the time period of mycobacterial shedding by an animal. In the context of the immunogenic compositions described herein, the terms encompass an amount of an immunogen which will induce an immunological response as described herein, either for antibody production or for control and/or prevention of infection.

By “mammalian subject” is meant any member of the class Mammalia, including, without limitation, humans and all other mammary gland-possessing animals (both male and female), such as humans and non-human primates; ruminants, including, but not limited to, bovine (e.g., cows, buffalo, and bison), ovine (e.g., sheep and goats), cervids (e.g., elk and deer), and camelids (e.g., camels and llamas); leporidae (e.g., rabbits and hares); porcine species (e.g., pigs and boar); domestic animals (e.g., cats and dogs); and wild carnivores and omnivores. The term does not denote a particular age. Thus, adults, newborns, and fetuses are intended to be covered.

### 2. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based in part on the discovery of mycobacterial antigens, e.g., MAP and M. bovis antigens, for use in vaccine compositions and diagnostics.

MAP causes a chronic, progressive granulomatous enteritis known as Johne's disease, or paratuberculosis, in ruminants and other mammals. The disease is endemic in many parts of the world and is responsible for considerable losses to the livestock and associated industries. Humans can be infected through MAP shedding into faeces and milk, and the infection can contribute to the etiology of inflammatory bowel disease (IBD), Crohn's disease, and other chronic gastrointestinal disorders. Diagnosis and control are problematic, in part due to the long incubation period of the disease when infected animals show no clinical signs and are difficult to detect, as well as due to the ability of the organism to survive and persist in the environment.

MAP has been isolated from a diverse range of both ruminant and non-ruminant hosts. There are two major groups of strains known as “Sheep-type” or “Type S,” and “Cattle-type” or “Type C,” originally named based on the host species from which they were first isolated. In addition to sheep, Type S strains include strains derived from other ovine species, as well as camelid isolates that are of a genetically distinct subtype. Another group of genetically distinct strains, termed “Bison” or “B-type,” has also been identified. See, e.g., Whittington et al., Mol. Cell Probes (2001) 15:139-145; Sohal et al., Microbiol. Res. (2010) 165:163-171. Human MAP isolates from patients with IBD appear to be part of the Type C group. See, e.g., Wynne et al., PLOS One (2011) 6e: 22171; Hsu et al., Front Microbiol. (2011) 2:236. For a detailed description of MAP strains, see, e.g., Stevenson, K., Vet. Res. (2015) 46:64.

M. bovis is the main causative agent of bovine tuberculosis (TB). Bovine TB causes important losses in the cattle industry, as the current means of controlling the disease is a “test and slaughter” approach where animals with positive skin reactions to crude preparations of mycobacterial antigens are identified as infected, and culled (Garnier et al. Proc. Natl. Acad. Sci. USA (2003) 100:7877-7882). M. bovis also affects humans and non-human primates, domestic animals (e.g. swine, sheep, goats, horses, camels, cats and dogs) and wild animals (cervids including water buffalo, bison, foxes, coyotes, common brushtail opossums, mustelids and rodents) (Cosivi et al., Emerg. Infect. Dis. (1998) 4:59-70). As described by Palmer et al. (Veterinary Medicine International (2012), article ID 236205), it is generally accepted that among wildlife species, such as the badger in the United Kingdom and the Republic of Ireland, the brushtail opossum in New Zealand, the European wild boar in the Iberian Peninsula, and the white-tailed deer in Michigan, United States, represent true maintenance hosts and a source of infection for other species. Zoonotic transmission of M. bovis occurs primarily through close contact with infected cattle or consumption of contaminated animal products (Müller et al., Emerg. Infect. Dis. (2013) 19 (6): 899-908)

The inventors have discovered immunogenic mycobacterial molecules using a reverse vaccinology approach. These molecules include one or more epitopes for stimulating an immune response in a subject of interest. One or more of the molecules can be provided in an isolated form as discrete components, or as fusion proteins. Antigens can be incorporated into pharmaceutical compositions, such as vaccine compositions, as well as into diagnostics.

The present invention thus provides immunological compositions and methods for controlling and/or preventing mycobacterial infections, such as, but not limited to, MAP, M. bovis, and Mtb infections, as well as for diagnosing MAP and M. bovis infection. Immunization can be achieved by any of the methods known in the art including, but not limited to, use of vaccines containing one or more isolated mycobacterial antigens, or fusion proteins comprising multiple antigens, or by passive immunization using antibodies directed against the antigens. Such methods are described in detail below. Moreover, the antigens described herein can be used for detecting the presence of mycobacterial infection, such as MAP and/or M. bovis infection, for example in a biological sample from a mammalian subject.

The vaccines are useful in mammalian subjects that are susceptible to mycobacterial infections, such as, but not limited to, MAP, M. bovis and Mtb infections, including without limitation, humans, non-human primates, bovine, sheep, goats, camelids, cervids, rabbits, hares, and any other mammal that might be in danger of infection, such as through shedding of the Mycobacterium in milk or faeces, or transmission of the Mycobacterium through exhaled air, sputum, urine, faeces, and pus, including direct contact with infected animals, contact with the secretions and/or excreta of an infected animal, or inhalation of aerosols, depending on the species involved.

In order to further an understanding of the invention, a more detailed discussion is provided below regarding MAP and M. bovis antigens, production thereof, compositions comprising the same, and methods of using such compositions in the control and/or prevention of mycobacterial infection, as well as in the diagnosis of infection. It is to be understood that the methods and compositions herein, while illustrated using MAP and M. bovis antigens, can also be applied to homologs and orthologs of these antigens from other mycobacteria.

A. MAP and M. bovis Antigens

Antigens for use in the subject compositions can be derived from any of several MAP and M. bovis strains and isolates. As explained herein, MAP is capable of infecting numerous mammals, including humans, and infection can cause a number of gastrointestinal diseases. M. bovis is also capable of infecting numerous mammals, including humans, and is the causative agent of tuberculosis and resultant pulmonary disorders in cattle. M. bovis can jump the species barrier and cause tuberculosis-like infection in humans and other mammals. These mycobacteria therefore have profound economic impacts on the animal industry, as well as posing danger to humans.

Tables 1, 3 and 4 in the examples show representative antigens for use in compositions for stimulating immune responses against MAP. As shown in Tables 3 and 4, several molecules listed in Table 1 have been identified as immunogenic. In addition to those molecules listed in Tables 1, 3 and 4, MAP antigens shown in Table 6, below (described in Facciuolo et al., Clin Vaccine Immunol (2013) 20:1783-1791) will also find use in compositions described herein.

TABLE 6
Additional representative MAP antigens.

| KEGG | NCBI | NCBI | NCBI Locus Tag | |
|---|---|---|---|---|
| entry | Protein GI | Locus tag | Gene Description | Localization_PSORTbv3 |
| MAP1693c | 41396145 | MAP_RS08610 | FKBP-type peptidyl-prolyl | |
| | | | cis-trans isomerase | |
| MAP1718c | 41396170 | MAP_RS08740 | Hypothetical protein | |
| MAP0196c | 41394642 | MAP_RS00985 | Hypothetical protein | |
| MAP3634 | 41398564 | MAP_RS18645 | Hypothetical protein | |
| MAP3428c | 41398357 | MAP_RS17625 | Cutinase | |
| MAP2785c | 41397242 | MAP_RS14245 | Hypothetical protein | |
| MAP2786c | 41397243 | MAP_RS14250 | Hypothetical protein | |
| MAP1981c | 41396434 | MAP_RS10065 | Hypothetical protein | |
| MAP1569 | 41396020 | MAP_RS07985 | Hypothetical protein | |
| MAP0471 | 41394918 | MAP_RS02415 | Uncharacterized protein | Periplasmic |

Tables 2 and 5 in the examples show representative antigens for use in compositions for stimulating immune responses against M. bovis. As shown in Table 5, several molecules listed in Table 2 were identified as immunogenic. MAP and M. bovis share thousands of genes encoding homologous proteins and can cause disease in some of the same hosts (e.g., ruminants and humans). As shown in the tables, several MAP and M. bovis antigens identified herein are orthologous, and several of the identified M. bovis antigens are orthologs of M. tuberculosis (Mtb) proteins. Hence, these antigens may provide cross-reactive antibodies to induce protective immune responses against MAP infection, M. bovis infection, and/or Mtb infection, as well as against infection caused by other mycobacterial species sharing homologous or orthologous proteins. Known orthologs to the M. bovis and MAP antigens, as well as orthologs identified in the tables (see, Tables 2 and 5) will therefore also find use in compositions as described herein.

Preferably, the subject compositions include one or more of these antigens, such as one, two, three, four, five, six, seven, eight, nine, ten, or more of the antigens in any combination, as well as antigens from other MAP and/or M. bovis strains or isolates that correspond to the antigens listed in the tables. Moreover, the antigens present in the compositions can include the full-length amino acid sequences, or fragments or variants of these sequences, so long as the antigens stimulate an immunological response, preferably, a neutralizing and/or protective immune response. Thus, the antigens can be provided with deletions from the N- or C-termini which do not disrupt immunogenicity, including without limitation, deletions of an N-terminal methionine if present, deletions of all or part of the transmembrane domain(s) if present, deletions of all or part of the cytoplasmic domain(s) if present, and deletions of the native signal sequence if present. Additionally, the molecules can include other N-terminal, C-terminal and internal deletions of amino acids or sequences irrelevant to immunogenicity. Moreover, the molecules can include additions, such as the presence of a heterologous signal sequence if desired, as well as amino acid linkers, and/or ligands useful in protein purification, such as histidine tags, glutathione-S-transferase or staphylococcal protein A.

It is to be understood that the present invention is not limited to the representative proteins described in the tables as a number of strains and isolates of these pathogens are known, and the corresponding proteins from these strains and isolates are intended to be captured herein.

As explained above, any of these antigens, as well as the corresponding antigens from different mycobacterial species, strains or isolates, can be used alone or in combination in the immunogenic compositions described herein, to provide protection against mycobacterial infection, such as, but not limited to, MAP, M. bovis and/or Mtb infection. The compositions can include antigens from more than one species, strain, or isolate. Thus, each of the components of a subunit composition or fusion protein can be obtained from the same MAP and/or M. bovis strain or isolate, or from different strains or isolates.

If more than one mycobacterial antigen is present in the immunogenic compositions, the compositions can include discrete antigens, i.e., isolated and purified antigens provided separately, or can include fusions of the desired antigens. The fusions will include two or more immunogenic mycobacterial proteins, such as two, three, four, five, six, seven, eight, nine, ten, etc., such as one or more of the mycobacterial antigens described herein, or antigens from other mycobacterial strains or isolates that correspond to the antigens described herein. Moreover, as explained above, the antigens present in the fusions can include the full-length amino acid sequences, or fragments or variants of these sequences so long as the antigens stimulate an immunological response, preferably, a protective immune response. At least one epitope from these antigens will be present. In some embodiments, the fusions will include repeats of desired epitopes. The antigens present in fusions can be derived from the same species, strain or isolate, or from different species, strains or isolates, to provide increased protection against a broad range of mycobacteria.

In certain embodiments, fusion proteins are provided that include multiple antigens, such as more than one epitope from a particular antigen, and/or epitopes from more than one antigen. The epitopes can be provided as the full-length antigen sequence, or in a partial sequence that includes the epitope. The epitopes can be from the same mycobacterial species, strain or isolate, or different species, strains or isolates. Additionally, the epitopes can be derived from the same mycobacterial protein or from different mycobacterial proteins from the same or different mycobacterial strain or isolate.

More particularly, chimeric fusion proteins may comprise multiple epitopes, a number of different proteins from the same or different species, strains or isolates, as well as multiple or tandem repeats of selected mycobacterial antigen sequences, multiple or tandem repeats of selected mycobacterial epitopes, or any conceivable combination thereof. Epitopes may be identified using techniques as described herein, or fragments of proteins may be tested for immunogenicity and active fragments used in compositions in lieu of the entire polypeptide. Fusions may also include the full-length sequence.

If multiple antigen sequences are present in fusions, they may be separated by spacers. A selected spacer sequence may encode a wide variety of moieties of one or more amino acids in length. Selected spacer groups may also provide enzyme cleavage sites so that an expressed chimeric molecule can be processed by proteolytic enzymes in vivo to yield a number of peptides.

For example, amino acids can be used as spacer sequences. Such spacers will typically include from 1-500 amino acids, such as 1-100 amino acids, e.g., 1-50 amino acids, such as 1-25 amino acids, 1-10 amino acids, 1-5 amino acids, or any integer between 1-500. The spacer amino acids may be the same or different between the various antigens. Particularly preferred amino acids for use as spacers are amino acids with small side groups, such as serine, alanine, glycine and valine. Various combinations of amino acids or repeats of the same amino acid may be used.

In order to enhance immunogenicity of the mycobacterial proteins, as well as multiple antigen fusion molecules, they may be conjugated with a carrier. By “carrier” is meant any molecule which when associated with an antigen of interest, imparts immunogenicity to the antigen. Examples of suitable carriers include large, slowly metabolized macromolecules such as: proteins; polysaccharides, such as sepharose, agarose, cellulose, cellulose beads and the like; polymeric amino acids such as polyglutamic acid, polylysine, and the like; amino acid copolymers; inactive virus particles; bacterial toxins such as tetanus toxoid, serum albumins, keyhole limpet hemocyanin, thyroglobulin, ovalbumin, sperm whale myoglobin, and other proteins well known to those skilled in the art.

These carriers may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl) propionate. Suitable carriers may also be modified to incorporate spacer arms (such as hexamethylene diamine or other bifunctional molecules of similar size) for attachment of peptides.

Additionally, the mycobacterial proteins and multiple antigen fusion molecules can be fused to either the carboxyl or amino terminals or both of the carrier molecule, or at sites internal to the carrier.

Carriers can be physically conjugated to the proteins of interest, using standard coupling reactions. Alternatively, chimeric molecules can be prepared recombinantly for use in the present invention, such as by fusing a gene encoding a suitable polypeptide carrier to one or more copies of a gene, or fragment thereof, encoding for selected mycobacterial proteins or mycobacterial multiple epitope fusion molecules.

The above-described antigens, fusions and carrier conjugates, can be produced recombinantly. A polynucleotide encoding these proteins can be introduced into an expression vector which can be expressed in a suitable expression system. A variety of bacterial, yeast, mammalian and insect expression systems are available in the art and any such expression system can be used. Optionally, a polynucleotide encoding these proteins can be translated in a cell-free translation system. Such methods are well known in the art. The proteins also can be constructed by solid phase protein synthesis.

B. MAP and M. bovis Polynucleotides

MAP and M. bovis polynucleotides encoding the MAP and M. bovis antigens for use in the subject compositions can be derived from any MAP or M. bovis strain or isolate. The polynucleotides can be modified for expression in a particular host cell, such as E coli. In addition, optimized MAP and M. bovis genes can be created by reverse engineering using the known amino acid sequences of the selected vaccine antigens and the codon preferences of the selected host cell. By synthesizing the encoding gene using standard oligonucleotide synthesis methods, and tagging the sequences to enable manipulations using the Gateway method, one can readily create an expression construct that will enable optimized production of the antigen in question in a suitable heterologous host, such as, but not limited to, Mycobacterium smegmatis, E. coli, Bacillus subtilis, Saccharomyces cerevisiae and/or Pichia pastoris, or other host, readily known to one of ordinary skill in the art and described below.

The polynucleotide sequences encoding MAP and M. bovis antigens will encode the full-length amino acid sequences, or fragments or variants of these sequences so long as the resulting antigens stimulate an immunological response, preferably, a protective immune response. Thus, the polynucleotides can encode antigens with deletions or additions, as described above.

Once the coding sequences for the desired antigens have been isolated or synthesized, they can be cloned into any suitable vector or replicon for expression. Numerous cloning vectors are known to those of skill in the art, and the selection of an appropriate cloning vector is a matter of choice. A variety of bacterial, yeast, plant, mammalian and insect expression systems are available in the art and any such expression system can be used. Optionally, a polynucleotide encoding these proteins can be translated in a cell-free translation system. Such methods are well known in the art.

Examples of recombinant DNA vectors for cloning and host cells which they can transform include the bacteriophage 2 (E. coli), pBR322 (E. coli), pACYC177 (E. coli), pET301/CT-Dest (E. coli), pKT230 (Gram-negative bacteria), pGV1106 (Gram-negative bacteria), pLAFR1 (Gram-negative bacteria), pME290 (non-E. coli Gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, Sambrook et al., Molecular Cloning, a laboratory manual, Cold Spring Harbor Laboratories, New York.

Insect cell expression systems, such as baculovirus systems, can also be used and are known to those of skill in the art. Plant expression systems can also be used to produce the immunogenic proteins. Generally, such systems use virus-based vectors to transfect plant cells with heterologous genes.

Viral systems, such as a vaccinia based infection/transfection system, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation product(s).

The coding sequence can be placed under the control of a promoter, ribosome binding site (for bacterial expression) and, optionally, an operator (collectively referred to herein as “control elements”), so that the DNA sequence encoding the desired antigen is transcribed into RNA in the host cell transformed by a vector containing this expression construction. The coding sequence may or may not contain a signal peptide or leader sequence. Leader sequences can be removed by the host in post-translational processing.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., to maintain the proper reading frame. It may also be desirable to produce mutants or analogs of the immunogenic proteins. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., Molecular Cloning, a laboratory manual, Cold Spring Harbor Laboratories, New York.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney (HEK) 293 cells, human hepatocellular carcinoma cells (e.g., Hep G2), as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host cell selected, the proteins of the present invention are produced by growing host cells transformed by an expression vector described above under conditions whereby the protein of interest is expressed. The selection of the appropriate growth conditions is within the skill of the art. If the proteins are not secreted, the cells are then disrupted, using chemical, physical or mechanical means, which lyse the cells yet keep the proteins substantially intact. Following disruption of the cells, cellular debris is removed, generally by centrifugation. Whether produced intracellularly or secreted, the protein can be further purified, using standard purification techniques such as but not limited to, column chromatography, ion-exchange chromatography, size-exclusion chromatography, electrophoresis, high-performance liquid chromatography (HPLC), immunoadsorbent techniques, affinity chromatography, immunoprecipitation, and the like.

### C. Antibodies

The antigens of the present invention can be used to produce antibodies for therapeutic (e.g., passive immunization), diagnostic and purification purposes. These antibodies can be polyclonal or monoclonal antibody preparations, monospecific antisera, or may be hybrid or chimeric antibodies, such as humanized antibodies, altered antibodies, F(ab′)₂ fragments, F(ab) fragments, Fv fragments, single-domain antibodies, dimeric or trimeric antibody fragment constructs, minibodies, or functional fragments thereof which bind to the antigen in question. Antibodies are produced using techniques well known to those of skill in the art.

### D. Compositions

Mycobacterial antigens, such as, but not limited to, MAP and M. bovis molecules, can be formulated into compositions for delivery to subjects for eliciting an immune response, such as for inhibiting infection. Compositions of the invention may comprise or be co-administered with non-MAP and/or non-M. bovis antigens, or with a combination of MAP and/or M. bovis antigens, as described herein. Methods of preparing such formulations are described in, e.g., Remington's Pharmaceutical Sciences, Mack Publishing Company, Easton, Pennsylvania, 22nd Edition, 2012. The compositions of the present invention can be prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Adjuvants which enhance the effectiveness of the composition may also be added to the formulation. Such adjuvants include any compound or combination of compounds that act to increase an immune response to the mycobacterial antigens, e.g., a MAP or M. bovis antigen or combination of antigens, thus reducing the quantity of antigen necessary in the vaccine, and/or the frequency of injection necessary in order to generate an adequate immune response.

For example, a triple adjuvant formulation as described in, e.g., U.S. Pat. No. 9,061,001, incorporated herein by reference in its entirety, can be used in the subject compositions. The triple adjuvant formulation includes a host defense peptide, in combination with a polyanionic polymer such as a polyphosphazene, and a nucleic acid sequence possessing immunostimulatory properties (ISS), such as an oligodeoxynucleotide molecule with or without a CpG motif (a cytosine followed by guanosine and linked by a phosphate bond) or the synthetic dsRNA analog poly(I:C).

Examples of host defense peptides for use in the combination adjuvant, as well as individually with the antigen include, without limitation, HH2 (VQLRIRVAVIRA-NH₂); 1002 (VQRWLIVWRIRK-NH₂); 1018 (VRLIVAVRIWRR-NH₂); Indolicidin (ILPWKWPWWPWRR-NH₂); HH111 (ILKWKWPWWPWRR-NH₂); HH113 (ILPWKKPWWPWRR-NH₂); HH970 (ILKWKWPWWKWRR-NH₂); HH1010 (ILRWKWRWWRWRR-NH₂); Nisin Z (Ile-Dhb-Ala-Ile-Dha-Leu-Ala-Abu-Pro-Gly-Ala-Lys-Abu-Gly-Ala-Leu-Met-Gly-Ala-Asn-Met-Lys-Abu-Ala-Abu-Ala-Asn-Ala-Ser-Ile-Asn-Val-Dha-Lys); JK1 (VFLRRIRVIVIR-NH₂); JK2 (VFWRRIRVWVIR-NH₂); JK3 (VQLRAIRVRVIR-NH₂); JK4 (VQLRRIRVWVIR-NH₂); JK5 (VQWRAIRVRVIR-NH₂); and JK6 (VQWRRIRVWVIR-NH₂). Any of the above peptides, as well as fragments and analogs thereof, that display the appropriate biological activity, such as the ability to modulate an immune response, such as to enhance an immune response to a co-delivered antigen, will find use herein.

Exemplary, non-limiting examples of ISSs for use in the triple adjuvant composition, or individually include, CpG oligonucleotides or non-CpG molecules. By “CpG oligonucleotide” or “CpG ODN” is meant an immunostimulatory nucleic acid containing at least one cytosine-guanine dinucleotide sequence (i.e., a 5′ cytidine followed by 3′ guanosine and linked by a phosphate bond) and which activates the immune system. An “unmethylated CpG oligonucleotide” is a nucleic acid molecule which contains an unmethylated cytosine-guanine dinucleotide sequence (i.e., an unmethylated 5′ cytidine followed by 3′ guanosine and linked by a phosphate bond) and which activates the immune system. A “methylated CpG oligonucleotide” is a nucleic acid which contains a methylated cytosine-guanine dinucleotide sequence (i.e., a methylated 5′ cytidine followed by a 3′ guanosine and linked by a phosphate bond) and which activates the immune system. CpG oligonucleotides are well known in the art and described in, e.g., U.S. Pat. Nos. 6,194,388; 6,207,646; 6,214,806; 6,218,371; 6,239,116; and 6,339,068; PCT Publication No. WO 01/22990; PCT Publication No. WO 03/015711; US Publication No. 20030139364, which patents and publications are incorporated herein by reference in their entireties.

Examples of such CpG oligonucleotides include, without limitation, 5′TCCATGACGTTCCTGACGTT3′, termed CpG ODN 1826, a Class B CpG; 5′TCGTCGTTGTCGTTTTGTCGTT3′, termed CpG ODN 2007, a Class B CpG; 5′TCGTCGTTTTGTCGTTTTGTCGTT3′, also termed CPG 7909 or 10103, a Class B CpG; 5′ GGGGACGACGTCGTGGGGGGG 3′, termed CpG 8954, a Class A CpG; and 5′TCGTCGTTTTCGGCGCGCGCCG 3′, also termed CpG 2395 or CpG 10101, a Class C CpG. All of the foregoing class B and C molecules are fully phosphorothioated.

Non-CpG oligonucleotides for use in the present composition include the double stranded polyriboinosinic acid: polyribocytidylic acid, also termed poly(I:C); and a non-CpG oligonucleotide 5′AAAAAAGGTACCTAAATAGTATGTTTCTGAAA3′.

Polyanionic polymers for use in the triple combination adjuvants or alone include polyphosphazenes (sometimes termed “polyphosphazines”). Typically, polyphosphazenes for use with the present adjuvant compositions will either take the form of a polymer in aqueous solution or a polymer microparticle, with or without encapsulated or adsorbed substances such as antigens or other adjuvants. For example, the polyphosphazene can be a soluble polyphosphazene, such as a polyphosphazene polyelectrolyte with ionized or ionizable pendant groups that contain, for example, carboxylic acid, sulfonic acid or hydroxyl moieties, and pendant groups that are susceptible to hydrolysis under conditions of use to impart biodegradable properties to the polymer. Such polyphosphazene polyelectrolytes are well known and described in, for example, U.S. Pat. Nos. 5,494,673; 5,562,909; 5,855,895; 6,015,563; and 6,261,573, incorporated herein by reference in their entireties. Alternatively, polyphosphazene polymers in the form of cross-linked microparticles will also find use herein. Such cross-linked polyphosphazene polymer microparticles are well known in the art and described in, e.g., U.S. Pat. Nos. 5,053,451; 5,149,543; 5,308,701; 5,494,682; 5,529,777; 5,807,757; 5,985,354; and 6,207,171, incorporated herein by reference in their entireties.

Examples of particular polyphosphazene polymers for use herein include poly[di(sodium carboxylatophenoxy)phosphazene] (PCPP) and poly(di-4-oxyphenylproprionate)phosphazene (PCEP), in various forms, such as the sodium salt, or acidic forms, as well as a polymer composed of varying percentages of PCPP or PCEP copolymer with hydroxyl groups, such as 90:10 PCPP/OH. Methods for synthesizing these compounds are known and described in the patents referenced above, as well as in Andrianov et al., Biomacromolecules (2004) 5:1999; Andrianov et al., Macromolecules (2004) 37:414; Mutwiri et al., Vaccine (2007) 25:1204.

Additional adjuvants include chitosan-based adjuvants, and any of the various saponins, oils, and other substances known in the art, such as AMPHIGEN™ which comprises de-oiled lecithin dissolved in an oil, usually light liquid paraffin. In vaccine preparations AMPHIGEN™ is dispersed in an aqueous solution or suspension of the immunizing antigen as an oil-in-water emulsion.

Compounds which may serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrimethylammonium bromide, while synthetic nonionic agents are exemplified by glyceryl esters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oil, or oil-in-water emulsions in which the oil component is mineral oil are preferred. Another oil component are the oil-in-water emulsions sold under the trade name of EMULSIGEN™, such as but not limited to EMULSIGEN PLUS™, comprising a light mineral oil as well as 0.05% formalin, and 30 μg/mL gentamicin as preservatives, available from MVP Laboratories, Ralston, NE. Also of use herein is an adjuvant known as “VSA3” which is a modified form of EMULSIGEN PLUS™ which includes DDA (See, U.S. Pat. No. 5,951,988, incorporated herein by reference in its entirety). The adjuvant MONTANIDE™ will also find use herein. Suitable animal oils include, for example, cod liver oil, halibut oil, menhaden oil, orange roughy oil and shark liver oil, all of which are available commercially. Suitable vegetable oils, include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include amines, quaternary ammonium compounds, guanidines, benzamidines and thiouroniums (Gall, D. (1966) Immunology 11:369 386). Specific compounds include dimethyldioctadecylammonium bromide (DDA) (available from Kodak) and N,N-dioctadecyl-N,N-bis(2-hydroxyethyl) propanediamine (“AVRIDINE”). See, e.g., U.S. Pat. No. 4,310,550, incorporated herein by reference in its entirety, which describes the use of N,N-higher alkyl-N′,N′-bis(2-hydroxyethyl) propane diamines in general, and AVRIDINE in particular, as vaccine adjuvants. U.S. Pat. No. 5,151,267 to Babiuk, incorporated herein by reference in its entirety, and Babiuk et al. (1986) Virology 159:57 66, also relate to the use of AVRIDINE as a vaccine adjuvant.

Other adjuvants are LPS, bacterial cell wall extracts, purified or synthetic cell wall components, inactivated bacterial cells, bacterial DNA, synthetic oligonucleotides and combinations thereof (Schijns et al., Curr. Opi. Immunol. (2000) 12:456), Mycobacterium phlei (M. phlei) cell wall extract (MCWE) (U.S. Pat. No. 4,744,984), M. phlei DNA (M-DNA), and M-DNA-M. phlei cell wall complex (MCC). In the context of the present invention, the adjuvants can contain inactivated mycobacterial cells, such as inactivated MAP and/or M. bovis cells; purified or crude extracts of a mycobacterial cell wall, such as a MAP or M. bovis cell wall; individual molecules purified from a mycobacterial cell wall, such as from a MAP or M. bovis cell wall; or even synthesized molecules to mimic components present within the cell wall.

Once prepared, the formulations will contain a “pharmaceutically effective amount” of the active ingredient, that is, an amount capable of achieving the desired response in a subject to which the composition is administered. In the control and/or prevention of a MAP disease, a “pharmaceutically effective amount” would preferably be an amount which prevents, reduces or ameliorates the symptoms of the disease in question. The exact amount is readily determined by one skilled in the art using standard tests. The active ingredient will typically range from about 1% to about 95% (w/w) of the composition, or even higher or lower if appropriate. With the present formulations, 1 μg to 2 mg, such as 10 μg to 1 mg, e.g., 25 μg to 0.5 mg, 50 μg to 300 μg, 100 μg to 250 μg, or any values between these ranges of active ingredient per mL of injected solution should be adequate to control and/or prevent infection when a dose of 1 to 5 mL per subject is administered. The quantity to be administered depends on the subject to be treated, the capacity of the subject's immune system to synthesize antibodies, and the degree of protection desired. Effective dosages can be readily established by one of ordinary skill in the art through routine trials establishing dose response curves.

The composition can be administered parenterally, e.g., by intratracheal, intramuscular, subcutaneous, intraperitoneal, or intravenous injection. The subject is administered at least one dose of the composition. Moreover, the subject may be administered as many doses as is required to bring about the desired biological effect.

Additional formulations which are suitable for other modes of administration include suppositories and, in some cases, aerosol, intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject antigens by the nasal mucosa.

Controlled or sustained release formulations are made by incorporating the antigen into carriers or vehicles such as liposomes (see, e.g., PCT/CA2019/051347, incorporated herein by reference in its entirety), nonresorbable impermeable polymers such as ethylenevinyl acetate copolymers and HYTREL copolymers, swellable polymers such as hydrogels, resorbable polymers such as collagen and certain polyacids or polyesters such as those used to make resorbable sutures, polyphosphazenes, alginate, microparticles, gelatin nanospheres, chitosan nanoparticles, and the like. The antigens described herein can also be delivered using implanted mini-pumps, well known in the art.

The vaccine can be administered to nursing mammals, such as nursing calves, as well as weaned mammals and adult mammals.

Prime-boost methods can be employed where one or more compositions are delivered in a “priming” step and, subsequently, one or more compositions are delivered in a “boosting” step. In certain embodiments, priming and boosting with one or more compositions described herein is followed by additional boosting. The compositions delivered can include the same antigens, or different antigens, given in any order and via any administration route.

### E. Tests to Determine the Efficacy of an Immune Response

One way of assessing efficacy of therapeutic treatment involves monitoring infection after administration of a composition of the invention. One way of assessing efficacy of prophylactic treatment involves monitoring immune responses against the mycobacterial antigens, such as MAP or M. bovis antigens, in the compositions of the invention after administration of the composition. Another way of assessing the immunogenicity of the immunogenic compositions of the present invention is to screen the subject's sera by immunoblot. A positive reaction indicates that the subject has previously mounted an immune response to the particular mycobacterial antigen, that is, the mycobacterial protein is an immunogen. This method may also be used to identify epitopes.

Another way of checking efficacy of therapeutic treatment involves monitoring infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against the antigens in the compositions of the invention after administration of the composition. Typically, serum-specific antibody responses are determined post-immunization but pre-challenge, whereas mucosal-specific antibody responses are determined post-immunization and post-challenge. The immunogenic compositions of the present invention can be evaluated in in vitro and in vivo animal models prior to host administration.

The efficacy of immunogenic compositions of the invention can also be determined in vivo by challenging animal models of infection with the immunogenic compositions. The immunogenic compositions may or may not be derived from the same strains as the challenge strains. Preferably the immunogenic compositions are derivable from the same strains as the challenge strains.

The immune response may be one or both of a TH1 immune response and a TH2 response. The immune response may be an improved or an enhanced or an altered immune response. The immune response may be one or both of a systemic and a mucosal immune response. An enhanced systemic and/or mucosal immunity is reflected in an enhanced TH1 and/or TH2 immune response. Preferably, the enhanced immune response includes an increase in the production of IgG1 and/or IgG2a and/or IgA. Preferably the mucosal immune response is a TH2 immune response. Preferably, the mucosal immune response includes an increase in the production of IgA.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

A TH1 immune response may include one or more of an increase in CTLs, an increase in one or more of the cytokines associated with a TH1 immune response (such as IL-2, IFNγ, and TNFβ), an increase in activated macrophages, an increase in NK activity, or an increase in the production of IgG2a. Preferably, the enhanced TH1 immune response will include an increase in IgG2a production.

The immunogenic compositions of the invention will preferably induce long lasting immunity that can quickly respond upon exposure to one or more infectious antigens.

### F. Diagnostic Assays

As explained above, the mycobacterial proteins, such as MAP and M. bovis proteins, variants, immunogenic fragments and fusions thereof, may also be used as diagnostics to detect the presence of reactive antibodies of e.g., MAP and/or M. bovis, in a biological sample in order to determine the presence of infection. For example, the presence of antibodies reactive with a mycobacterial protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation; PCR-based assays, etc. The reactions generally include revealing labels such as fluorescent, chemiluminescent, radioactive, enzymatic labels or dye molecules, or other methods for detecting the formation of a complex between the antigen and the antibody or antibodies reacted therewith. These assays can also be used to differentiate infected animals from vaccinated animals (DIVA) in order to remove infected animals from food production.

The aforementioned assays generally involve separation of unbound antibody in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. Typically, a solid support is first reacted with a solid phase component (e.g., one or more MAP or M. bovis proteins or fusions) under suitable binding conditions such that the component is sufficiently immobilized to the support. Sometimes, immobilization of the antigen to the support can be enhanced by first coupling the antigen to a protein with better binding properties. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other molecules that can be used to bind the antigens to the support include polysaccharides, polylactic acids, polyglycolic acids, polymeric amino acids, amino acid copolymers, and the like. Such molecules and methods of coupling these molecules to the antigens, are well known to those of ordinary skill in the art. See, e.g., Brinkley, M. A. Bioconjugate Chem. (1992) 3:2-13; Hashida et al., J. Appl. Biochem. (1984) 6:56-63; and Anjaneyulu and Staros, International J. of Peptide and Protein Res. (1987) 30:117-124.

After reacting the solid support with the solid phase component, any non-immobilized solid-phase components are removed from the support by washing, and the support-bound component is then contacted with a biological sample suspected of containing ligand moieties (e.g., antibodies toward the immobilized antigens) under suitable binding conditions. After washing to remove any non-bound ligand, a secondary binder moiety is added under suitable binding conditions, wherein the secondary binder is capable of associating selectively with the bound ligand. The presence of the secondary binder can then be detected using techniques well known in the art.

More particularly, an ELISA method can be used, wherein the wells of a microtiter plate are coated with a mycobacterial protein or fusion, such as a MAP and/or M. bovis antigen or fusion. A biological sample containing or suspected of containing, for example, anti-MAP and/or anti-M. bovis immunoglobulin molecules, is then added to the coated wells. After a period of incubation sufficient to allow antibody binding to the immobilized antigen, the plate(s) can be washed to remove unbound moieties and a detectably labeled secondary binding molecule added. The secondary binding molecule is allowed to react with any captured sample antibodies, the plate washed and the presence of the secondary binding molecule detected using methods well known in the art.

Thus, in one particular embodiment, the presence of bound anti-MAP and/or anti-M. bovis ligands from a biological sample can be readily detected using a secondary binder comprising an antibody directed against the antibody ligands. A number of immunoglobulin (Ig) molecules are known in the art which can be readily conjugated to a detectable enzyme label, such as horseradish peroxidase, alkaline phosphatase or urease, using methods known to those of skill in the art. An appropriate enzyme substrate is then used to generate a detectable signal. In other related embodiments, competitive-type ELISA techniques can be practiced using methods known to those skilled in the art.

Assays can also be conducted in solution, such that the mycobacterial proteins and antibodies specific for those proteins form complexes under precipitating conditions. In one particular embodiment, MAP or M. bovis proteins can be attached to a solid phase particle (e.g., an agarose bead or the like) using coupling techniques known in the art, such as by direct chemical or indirect coupling. The antigen-coated particle is then contacted under suitable binding conditions with a biological sample suspected of containing antibodies for the mycobacterial proteins. Cross-linking between bound antibodies causes the formation of particle-antigen-antibody complex aggregates which can be precipitated and separated from the sample using washing and/or centrifugation. The reaction mixture can be analyzed to determine the presence or absence of antibody-antigen complexes using any of a number of standard methods, such as those immunodiagnostic methods described above.

In yet a further embodiment, an immunoaffinity matrix can be provided, wherein a polyclonal population of antibodies from a biological sample suspected of containing anti-MAP molecules and/or anti-M. bovis molecules, is immobilized to a substrate. In this regard, an initial affinity purification of the sample can be carried out using immobilized antigens. The resultant sample preparation will thus only contain anti-MAP and/or anti-M. bovis moieties, avoiding potential nonspecific binding properties in the affinity support. A number of methods of immobilizing immunoglobulins (either intact or in specific fragments) at high yield and good retention of antigen binding activity are known in the art.

Accordingly, once the immunoglobulin molecules have been immobilized to provide an immunoaffinity matrix, labeled mycobacterial proteins are contacted with the bound antibodies under suitable binding conditions. After any non-specifically bound antigen has been washed from the immunoaffinity support, the presence of bound antigen can be determined by assaying for label using methods known in the art.

Additionally, antibodies raised to the mycobacterial protein, such as MAP or M. bovis proteins, rather than the proteins themselves, can be used in the above-described assays in order to detect the presence of antibodies to the proteins in a given sample. These assays are performed essentially as described above and are well known to those of skill in the art.

PCR-based assays can also be used to detect the presence of mycobacterial infection in a biological sample. Real-time or quantitative PCR (qPCR) methods can be conducted using fluorescently-labeled specific oligonucleotide probes and monitoring the fluorescence after each cycle.

### G. Kits

The invention also provides kits comprising one or more containers of compositions of the invention. Compositions can be in liquid form or can be lyophilized, as can individual antigens. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may further include a third component comprising an adjuvant.

The kit can also comprise a package insert containing written or computer-readable instructions for methods of inducing immunity or for controlling infections. The package insert can be an unapproved draft package insert or can be a package insert approved by the Food and Drug Administration (FDA) or other regulatory body.

The invention also provides a delivery device pre-filled with the immunogenic compositions of the invention.

Similarly, antibodies can be provided in kits, with suitable instructions and other necessary reagents. The kit can also contain, depending on if the antibodies are to be used in immunoassays, suitable labels and other packaged reagents and materials (i.e. wash buffers and the like). Standard immunoassays can be conducted using these kits.

### 3. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Identification of Putative MAP Antigens Using Reverse Vaccinology

Reverse vaccinology approaches, combining immunoinformatics, bacterial comparative genomics, and transcriptomics, were used to identify putative MAP diagnostic and vaccine antigens. Table 1 shows putative MAP antigens identified from the MAP strain K10 annotated genome (NCBI Reference Sequence NC_022944.2). Ninety-two of these antigens were proteins predicted to have an extracellular, periplasmic or outer membrane localization. Of these 92 antigens, nine were moonlighting proteins, one was identified as a non-cytoplasmic protein, and one was identified as a cytoplasmic membrane protein (see, Table 1). Other antigens in Table 1 were identified by bacterial transcriptional profiling of MAP-infected bovine CD14+ monocytes using methods described in, e.g., Arsenault et al., Infect. Immunol. (2012) 80:3039-3048. Additional putative MAP antigens in Table 1 were homologs of Mycobacterium tuberculosis (Mtb) proteins.

TABLE 1
Putative MAP antigens.

| KEGG | NCBI | NCBI | NCBI Locus Tag | |
|---|---|---|---|---|
| entry | Protein GI | Locus tag | Gene Description | Localization_PSORTbv3 |
| MAP0011* | 41394457 | MAP_RS00065 | Peptidyl-prolyl cis-trans | Cytoplasmic |
| | | | isomerase | |
| MAP0064 | 41394510 | MAP_RS00335 | Penicillin-binding protein | Unknown |
| MAP0109* | 41394555 | MAP_RS00560 | MCE family protein | Unknown |
| MAP0110 | 41394556 | MAP_RS00565 | Hypothetical protein | Unknown |
| MAP0115¹ | 41394561 | MAP_RS00590 | Uncharacterized protein | Periplasmic |
| MAP0144¹ | 41394590 | MAP_RS00730 | Hypothetical protein | Cytoplasmic Membrane |
| MAP0145 | 41394591 | MAP_RS00735 | Hypothetical protein | Unknown |
| MAP0150c | 41394596 | MAP_RS00760 | Acyl-CoA dehydrogenase | Cytoplasmic |
| MAP0157 | 41394603 | MAP_RS00790 | Hypothetical protein | Unknown |
| MAP0162* | 41394608 | MAP_RS00815 | ESX secretion-associated | Cytoplasmic |
| | | | protein EspG | |
| MAP0183c | 41394629 | MAP_RS00920 | Uncharacterized protein | Periplasmic |
| MAP0187c*¹ | 41394633 | MAP_RS00940 | Superoxide dismutase | Periplasmic |
| MAP0214 | 41394660 | MAP_RS01075 | Hypothetical protein | Cytoplasmic Membrane |
| MAP0216* | 41394662 | MAP_RS01085 | Esterase family protein | Unknown |
| MAP0217*¹ | 41394663 | MAP_RS01090 | Esterase family protein | Extracellular |
| MAP0218 | 41394664 | MAP_RS01095 | Uncharacterized protein | Periplasmic |
| MAP0259* | 41394705 | MAP_RS01315 | DNA-binding response | Cytoplasmic |
| | | | regulator | |
| MAP0261c | 41394707 | MAP_RS01325 | Uncharacterized protein | Extracellular |
| MAP0273 | 41394719 | MAP_RS01385 | Hypothetical protein | Cytoplasmic Membrane |
| MAP0280 | 41394726 | MAP_RS01425 | Glucanase | Extracellular |
| MAP0326 | 41394773 | MAP_RS01655 | Hypothetical protein | Periplasmic |
| MAP0333 | 41394780 | MAP_RS01690 | Hypothetical protein | Extracellular |
| MAP0367¹ | 41394814 | MAP_RS01870 | Uncharacterized protein | Periplasmic |
| MAP0401 | 41394848 | MAP_RS02045 | Hypothetical protein | Unknown |
| MAP0403* | 41394850 | MAP_RS02055 | Serine protease | Cytoplasmic Membrane |
| MAP0409 | 41394856 | MAP_RS02085 | ABC transporter substrate- | Periplasmic |
| | | | binding protein | |
| MAP0412* | 41394859 | MAP_RS02100 | ABC transporter ATP- | Cytoplasmic Membrane |
| | | | binding protein | |
| MAP0440c* | 41394887 | MAP_RS02250 | Hypothetical protein | Periplasmic |
| MAP0474c | 41394921 | MAP_RS02430 | Hypothetical protein | Unknown |
| MAP0494¹ | 41394941 | MAP_RS02525 | 3-beta hydroxysteroid | Unknown |
| | | | dehydrogenase | |
| MAP0498¹ | 41394945 | MAP_RS02545 | Hydrolase | Extracellular |
| MAP0567 | 41395014 | MAP_RS02885 | MCE family protein | Extracellular |
| MAP0572c | 41395019 | MAP_RS02910 | Uncharacterized protein | Periplasmic |
| MAP0574 | 41395021 | MAP_RS02920 | Phosphate ABC transporter | Cytoplasmic Membrane |
| | | | ATP-binding protein | |
| MAP0584 | 41395032 | MAP_RS02970 | Uncharacterized protein | Extracellular |
| MAP0592* | 41395040 | MAP_RS03015 | Two-component sensor | Cytoplasmic Membrane |
| | | | histidine kinase | |
| MAP0615 | 41395063 | MAP_RS03130 | S9 family peptidase | Periplasmic |
| MAP0645c | 41395093 | MAP_RS03280 | Sulfurtransferase | Periplasmic |
| MAP0651 | 41395099 | MAP_RS03310 | Phosphate ABC transporter | Periplasmic |
| | | | substrate-binding protein PstS | |
| MAP0701c | 41395149 | MAP_RS03580 | Oxidase | Periplasmic |
| MAP0728 | 41395176 | MAP_RS03725 | Uncharacterized protein | Periplasmic |
| MAP0750c¹ | 41395198 | MAP_RS03835 | Uncharacterized protein | Outer Membrane |
| MAP0785 | 41395233 | MAP_RS04015 | CoA transferase | Cytoplasmic |
| MAP0805c | 41395253 | MAP_RS04115 | Hypothetical protein | Unknown |
| MAP0872 | 41395320 | MAP_RS04420 | Phosphate ABC transporter | Unknown |
| | | | substrate-binding protein PstS | |
| MAP0896 | 41395345 | MAP_RS04540 | Succinate-CoA ligase subunit | Cytoplasmic |
| | | | beta | |
| MAP0900* | 41395349 | MAP_RS04560 | Hypothetical protein | Cytoplasmic Membrane |
| MAP0918¹ | 41395367 | MAP_RS04650 | Serine protease | Periplasmic |
| MAP0951 | 41395400 | MAP_RS04815 | Uncharacterized protein | Outer Membrane |
| MAP0974* | 41395423 | MAP_RS04935 | Resuscitation-promoting | Unknown |
| | | | factor | |
| MAP0981c | 41395430 | MAP_RS04970 | Hypothetical protein | Extracellular |
| MAP1018c | 41395467 | MAP_RS05190 | 3-hydroxyisobutyryl-CoA | Moonlighting proteins |
| | | | hydrolase | |
| MAP1050c* | 41395499 | MAP_RS05350 | Peptidyl-prolyl cis-trans | Unknown |
| | | | isomerase | |
| MAP1060c | 41395509 | MAP_RS05405 | Membrane protein | Extracellular |
| MAP1086 | 41395535 | MAP_RS05540 | Peptide ABC transporter | Periplasmic |
| | | | substrate-binding protein | |
| MAP1138c* | 41395587 | MAP_RS05795 | Hypothetical protein | Cytoplasmic Membrane |
| MAP1164 | 41395614 | MAP_RS05920 | Aldehyde dehydrogenase | Cytoplasmic |
| MAP1165 | 41395615 | MAP_RS05925 | Phosphoglycerate kinase | Moonlighting proteins |
| MAP1166 | 41395616 | MAP_RS05930 | Triosephosphate isomerase | Moonlighting proteins |
| MAP1174c | 41395624 | MAP_RS05970 | 6-phosphogluconol actonase | Moonlighting proteins |
| MAP1177c | 41395627 | MAP_RS05985 | Transaldolase | Moonlighting proteins |
| MAP1272c | 41395722 | MAP_RS06455 | Hypothetical protein | Unknown |
| MAP1327c | 41395777 | MAP_RS06740 | Uncharacterized protein | Extracellular |
| MAP1405 | 41395856 | MAP_RS07135 | Channel-forming protein | Outer Membrane |
| MAP1476c | 41395927 | MAP_RS07510 | Cutinase | Extracellular |
| MAP1507* | 41395958 | MAP_RS07675 | PE family protein | Periplasmic |
| MAP1509* | 41395960 | MAP_RS07690 | ESX secretion-associated | Cytoplasmic |
| | | | protein EspG | |
| MAP1530¹ | 41395981 | MAP_RS07800 | Hypothetical protein | Periplasmic |
| MAP1549c¹ | 41396000 | MAP_RS07885 | Malate synthase G | Moonlighting proteins |
| MAP1565 | 41396016 | MAP_RS07965 | Molybdate-binding protein | Periplasmic |
| MAP1588c¹ | 41396039 | MAP_RS08080 | Alkylhydroperoxidase | Moonlighting proteins |
| MAP1595 | 41396046 | MAP_RS08115 | Bacterioferritin | Cytoplasmic |
| MAP1607c* | 41396058 | MAP_RS08175 | Resuscitation-promoting | Non-cytoplasmic |
| | | | factor RpfC | (sigl sequence) |
| MAP1609c* | 41396060 | MAP_RS08185 | Esterase family protein | Extracellular |
| MAP1653 | 41396105 | MAP_RS08400 | 2-Cys peroxiredoxin | Periplasmic |
| MAP1680c | 41396132 | MAP_RS08540 | Cutinase family protein | Extracellular |
| MAP1725c | 41396177 | MAP_RS08780 | Catalase | Periplasmic |
| MAP1769c | 41396221 | MAP_RS09000 | Sugar ABC transporter | Periplasmic |
| | | | substrate-binding protein | |
| MAP1781* | 41396233 | MAP_RS09060 | Hypothetical protein | Unknown |
| MAP1784c | 41396236 | MAP_RS09075 | Uncharacterized protein | Extracellular |
| MAP1858 | 41396310 | MAP_RS09440 | Mannan-binding protein | Periplasmic |
| MAP1928c | 41396381 | MAP_RS09795 | Endopeptidase | Periplasmic |
| MAP1962 | 41396415 | MAP_RS09970 | Type I glutamate-ammonia | Cytoplasmic |
| | | | ligase | |
| MAP1966c | 41396419 | MAP_RS09990 | Type I glutamate-ammonia | Cytoplasmic |
| | | | ligase | |
| MAP1997 | 41396450 | MAP_RS10150 | Acyl carrier protein | Cytoplasmic |
| MAP2057 | 41396510 | MAP_RS10450 | Haloalkane dehalogenase | Periplasmic |
| MAP2064 | 41396517 | MAP_RS10485 | Sensor domain-containing | Extracellular |
| | | | protein | |
| MAP2069c | 41396522 | MAP_RS10510 | Molecular chaperone HtpG | Cytoplasmic |
| MAP2109c | 41396562 | MAP_RS10720 | Uncharacterized protein | Outer Membrane |
| MAP2120c | 41399240 | MAP_RS10780 | Cysteine desulfurase | Moonlighting proteins |
| MAP2121c¹ | 41396574 | MAP_RS10785 | Hypothetical protein | Cytoplasmic Membrane |
| MAP2167c | 41396620 | MAP_RS11025 | Hypothetical protein | Unknown |
| MAP2168c | 41396621 | MAP_RS11030 | Hypothetical protein | Unknown |
| MAP2213c | 41396667 | MAP_RS11255 | Sulfate ABC transporter | Periplasmic |
| | | | substrate-binding protein | |
| MAP2216c | 41396670 | MAP_RS11270 | Sensor domain-containing | Periplasmic |
| | | | protein | |
| MAP2268c | 41396722 | MAP_RS11540 | Nucleoside-diphosphate | Cytoplasmic |
| | | | kinase | |
| MAP2273c* | 41396727 | MAP_RS11565 | Resuscitation-promoting | Unknown |
| | | | factor RpfE | |
| MAP2283 | 41396737 | MAP_RS11625 | Esterase | Periplasmic |
| MAP2286c | 41396740 | MAP_RS11640 | DBSA oxidoreductase | Cytoplasmic |
| MAP2498c | 41396954 | MAP_RS12745 | Uncharacterized protein | Extracellular |
| MAP2506c | 41396962 | MAP_RS12780 | Uncharacterized protein | Periplasmic |
| MAP2523c | 41396979 | MAP_RS12865 | Uncharacterized protein | Periplasmic |
| MAP2552 | 41397008 | MAP_RS13020 | Lytic transglycosylase | Extracellular |
| MAP2555c* | 41397011 | MAP_RS13035 | Serine protease | Periplasmic |
| MAP2576c* | 41397032 | MAP_RS13145 | Hypothetical protein | Periplasmic |
| MAP2600 | 41397056 | MAP_RS13265 | PPE family protein | Extracellular |
| MAP2625 | 41397081 | MAP_RS13390 | Uncharacterized protein | Extracellular |
| MAP2626 | 41397082 | MAP_RS13395 | Uncharacterized protein | Periplasmic |
| MAP2698c* | 41397155 | MAP_RS13745 | Acyl-ACP desaturase | Unknown |
| MAP2744c | 41397201 | MAP_RS13985 | Catalase-related peroxidase | Periplasmic |
| MAP2746 | 41397203 | MAP_RS13995 | Hypothetical protein | Unknown |
| MAP2832 | 41397289 | MAP_RS14485 | Alpha/beta hydrolase | Extracellular |
| MAP2855c* | 41397312 | MAP_RS14610 | Hypothetical protein | Cytoplasmic |
| MAP2979 | 41397436 | MAP_RS15250 | D-alanyl-D-alanine | Periplasmic |
| | | | carboxypeptidase | |
| MAP3007 | 41397925 | MAP_RS15390 | Aldo/keto reductase | Cytoplasmic |
| MAP3092 | 41398010 | MAP_RS15855 | Iron citrate ABC transporter | Periplasmic |
| | | | substrate-binding protein | |
| MAP3184¹ | 41398102 | MAP_RS16340 | PPE family protein | Extracellular |
| MAP3236 | 41398154 | MAP_RS16615 | Catalase HPII | Cytoplasmic |
| MAP3298 | 41398227 | MAP_RS16950 | NrdH-redoxin | Periplasmic |
| MAP3362 | 41398291 | MAP_RS17285 | Adenosylhomocysteinase | Cytoplasmic |
| MAP3384* | 41398313 | MAP_RS17400 | Copper-translocating P-type | Cytoplasmic Membrane |
| | | | ATPase | |
| MAP3387c* | 41398316 | MAP_RS17415 | Serine/threonine protein | Periplasmic |
| | | | kinase | |
| MAP3417c¹ | 41398346 | MAP_RS17565 | Esterase | Extracellular |
| MAP3456c | 41398385 | MAP_RS17770 | Isocitrate dehydrogenase, | Cytoplasmic |
| | | | NADP-dependent | |
| MAP3495c | 41398424 | MAP_RS17970 | Cutinase | Unknown |
| MAP3527 | 41398456 | MAP_RS18120 | Serine protease | Periplasmic |
| MAP3531c* | 41398460 | MAP_RS18140 | Esterase family protein | Unknown |
| MAP3550¹ | 41398480 | MAP_RS18240 | Alpha/beta hydrolase | Extracellular |
| MAP3613 | 41398543 | MAP_RS18550 | Uncharacterized protein | Extracellular |
| MAP3638 | 41398568 | MAP_RS18665 | Hemophore | Periplasmic |
| MAP3651c | 41398581 | MAP_RS18725 | Acyl-CoA dehydrogenase | Cytoplasmic |
| MAP3775c | 41398705 | MAP_RS19350 | ABC transporter | Cytoplasmic Membrane |
| MAP3785* | 41398715 | MAP_RS19400 | ESX secretion-associated | Unknown |
| | | | protein EspG | |
| MAP3802c | 41398733 | MAP_RS19495 | Membrane protein | Extracellular |
| MAP3836c¹ | 41398767 | MAP_RS19675 | Uncharacterized protein | Extracellular |
| MAP3840*¹ | 41398771 | MAP_RS19695 | Molecular chaperone DnaK | Cytoplasmic |
| MAP3875c | 41398806 | MAP_RS19880 | Uncharacterized protein | Periplasmic |
| MAP3902c | 41398833 | MAP_RS20015 | Uncharacterized protein | Extracellular |
| MAP3906 | 41398837 | MAP_RS20035 | Amidohydrolase | Extracellular |
| MAP3939c*¹ | 41398870 | MAP_RS20205 | PPE family protein | Cytoplasmic Membrane |
| MAP3951c | 41398882 | MAP_RS20265 | S9 family peptidase | Periplasmic |
| MAP3968*¹ | 41398899 | MAP_RS20350 | Hypothetical protein | Cytoplasmic |
| MAP3970 | 41398901 | MAP_RS20360 | Uncharacterized protein | Periplasmic |
| MAP3972c | 41398903 | MAP_RS20370 | Uncharacterized protein | Extracellular |
| MAP4034 | 41398965 | MAP_RS20680 | Uncharacterized protein | Periplasmic |
| MAP4142¹ | 41399074 | MAP_RS21255 | Elongation factor G | Cytoplasmic |
| MAP4143*¹ | 41399075 | MAP_RS21260 | Elongation factor Tu | Cytoplasmic |
| MAP4144 | 41399076 | MAP_RS21265 | PE family protein | Periplasmic |
| MAP4172c | 41399104 | MAP_RS21410 | Phosphate ABC transporter | Periplasmic |
| | | | substrate-binding protein PstS | |
| MAP4199* | 41399131 | MAP_RS21545 | Adenylate kinase | Cytoplasmic |
| MAP4236c | 41399168 | MAP_RS21730 | Cutinase family protein | Extracellular |
| MAP4237c¹ | 41399169 | MAP_RS21735 | Cutinase | Extracellular |
| MAP4263¹ | 41399195 | MAP_RS21865 | tRNA (adenosine(37)-N6)- | Extracellular |
| | | | threonylcarbamoyltransferase | |
| | | | complex transferase subunit | |
| | | | TsaD | |
| MAP4265* | 41399197 | MAP_RS21875 | Molecular chaperone GroEL | Cytoplasmic |
| MAP4284* | 41399216 | MAP_RS21965 | Heavy metal translocating P-type | Cytoplasmic Membrane |
| | | | ATPase | |
| MAP4308c | 41396573 | MAP_RS22095 | Class I fructose-bisphosphate | Moonlighting proteins |
| | | | aldolase | |
| MAP4329c | 41399261 | MAP_RS22190 | Uncharacterized protein | Extracellular |
| MAP4339 | 41399271 | MAP_RS22255 | Thioredoxin-disulfide | Cytoplasmic |
| | | | reductase | |
| MAP4340* | 41399272 | MAP_RS22260 | Thiol reductase thioredoxin | Cytoplasmic |
| MAP4341 | 41399273 | MAP_RS22265 | N-acetymuramyl-L-alanine | Cytoplasmic |
| | | | amidase | |
| MAP4343c | 41399275 | MAP_RS22275 | Chromosome partitioning | Cytoplasmic |
| | | | protein ParB | |
| MAP0011* | 41394457 | MAP_RS00065 | Peptidyl-prolyl cis-trans | |
| | | | isomerase | |
| MAP0064 | 41394510 | MAP_RS00335 | Penicillin-binding protein | |
| MAP0109* | 41394555 | MAP_RS00560 | MCE family protein | |
| MAP0110 | 41394556 | MAP_RS00565 | Hypothetical protein | |
| MAP0115¹ | 41394561 | MAP_RS00590 | Uncharacterized protein | Periplasmic |
| MAP0144¹ | 41394590 | MAP_RS00730 | Hypothetical protein | |
| MAP0145 | 41394591 | MAP_RS00735 | Hypothetical protein | |
| MAP0150c | 41394596 | MAP_RS00760 | Acyl-CoA dehydrogenase | |
| MAP0157 | 41394603 | MAP_RS00790 | Hypothetical protein | |
| MAP0162* | 41394608 | MAP_RS00815 | ESX secretion-associated | |
| | | | protein EspG | |
| MAP0183c | 41394629 | MAP_RS00920 | Uncharacterized protein | Periplasmic |
| MAP0187c*¹ | 41394633 | MAP_RS00940 | Superoxide dismutase | |
| MAP0214 | 41394660 | MAP_RS01075 | Hypothetical protein | |
| MAP0216* | 41394662 | MAP_RS01085 | Esterase family protein | |
| MAP0217*¹ | 41394663 | MAP_RS01090 | Esterase family protein | Extracellular |
| MAP0218 | 41394664 | MAP_RS01095 | Uncharacterized protein | Periplasmic |
| MAP0259* | 41394705 | MAP_RS01315 | DNA-binding response | Cytoplasmic |
| | | | regulator | |
| MAP0261c | 41394707 | MAP_RS01325 | Uncharacterized protein | Extracellular |
| MAP0273 | 41394719 | MAP_RS01385 | Hypothetical protein | |
| MAP0280 | 41394726 | MAP_RS01425 | Glucanase | Extracellular |
| MAP0326 | 41394773 | MAP_RS01655 | Hypothetical protein | |
| MAP0333 | 41394780 | MAP_RS01690 | Hypothetical protein | |
| MAP0367¹ | 41394814 | MAP_RS01870 | Uncharacterized protein | Periplasmic |
| MAP0401 | 41394848 | MAP_RS02045 | Hypothetical protein | |
| MAP0403* | 41394850 | MAP_RS02055 | Serine protease | Cytoplasmic Membrane |
| MAP0409 | 41394856 | MAP_RS02085 | ABC transporter substrate- | Periplasmic |
| | | | binding protein | |
| MAP0412* | 41394859 | MAP_RS02100 | ABC transporter ATP- | |
| | | | binding protein | |
| MAP0440c* | 41394887 | MAP_RS02250 | Hypothetical protein | Periplasmic |
| MAP0474c | 41394921 | MAP_RS02430 | Hypothetical protein | |
| MAP0494¹ | 41394941 | MAP_RS02525 | 3-beta hydroxy steroid | |
| | | | dehydrogenase | |
| MAP0498¹ | 41394945 | MAP_RS02545 | Hydrolase | Extracellular |
| MAP0567 | 41395014 | MAP_RS02885 | MCE family protein | Extracellular |
| MAP0572c | 41395019 | MAP_RS02910 | Uncharacterized protein | Periplasmic |
| MAP0574 | 41395021 | MAP_RS02920 | Phosphate ABC transporter | |
| | | | ATP-binding protein | |
| MAP0584 | 41395032 | MAP_RS02970 | Uncharacterized protein | Extracellular |
| MAP0592* | 41395040 | MAP_RS03015 | Two-component sensor | |
| | | | histidine kinase | |
| MAP0615 | 41395063 | MAP_RS03130 | S9 family peptidase | Periplasmic |
| MAP0645c | 41395093 | MAP_RS03280 | Sulfurtransferase | Periplasmic |
| MAP0651 | 41395099 | MAP_RS03310 | Phosphate ABC transporter | Periplasmic |
| | | | substrate-binding protein PstS | |
| MAP0701c | 41395149 | MAP_RS03580 | Oxidase | Periplasmic |
| MAP0728 | 41395176 | MAP_RS03725 | Uncharacterized protein | Periplasmic |
| MAP0750c¹ | 41395198 | MAP_RS03835 | Uncharacterized protein | Outer Membrane |
| MAP0785 | 41395233 | MAP_RS04015 | CoA transferase | |
| MAP0805c | 41395253 | MAP_RS04115 | Hypothetical protein | |
| MAP0872 | 41395320 | MAP_RS04420 | Phosphate ABC transporter | |
| | | | substrate-binding protein PstS | |
| MAP0896 | 41395345 | MAP_RS04540 | Succinate-CoA ligase subunit | |
| | | | beta | |
| MAP0900* | 41395349 | MAP_RS04560 | Hypothetical protein | Cytoplasmic Membrane |
| MAP0918¹ | 41395367 | MAP_RS04650 | Serine protease | Periplasmic |
| MAP0951 | 41395400 | MAP_RS04815 | Uncharacterized protein | Outer Membrane |
| MAP0974* | 41395423 | MAP_RS04935 | Resuscitation-promoting | |
| | | | factor | |
| MAP0981c | 41395430 | MAP_RS04970 | Hypothetical protein | Extracellular |
| MAP1018c | 41395467 | MAP_RS05190 | 3-hydroxyisobutyryl-CoA | Moonlighting proteins |
| | | | hydrolase | |
| MAP1050c* | 41395499 | MAP_RS05350 | Peptidyl-prolyl cis-trans | |
| | | | isomerase | |
| MAP1060c | 41395509 | MAP_RS05405 | Membrane protein | Extracellular |
| MAP1086 | 41395535 | MAP_RS05540 | Peptide ABC transporter | Periplasmic |
| | | | substrate-binding protein | |
| MAP1138c* | 41395587 | MAP_RS05795 | Hypothetical protein | |
| MAP1164 | 41395614 | MAP_RS05920 | Aldehyde dehydrogenase | |
| MAP1165 | 41395615 | MAP_RS05925 | Phosphoglycerate kinase | Moonlighting proteins |
| MAP1166 | 41395616 | MAP_RS05930 | Triosephosphate isomerase | Moonlighting proteins |
| MAP1174c | 41395624 | MAP_RS05970 | 6-phosphogluconolactonase | Moonlighting proteins |
| MAP1177c | 41395627 | MAP_RS05985 | Transaldolase | Moonlighting proteins |
| MAP1272c | 41395722 | MAP_RS06455 | Hypothetical protein | |
| MAP1327c | 41395777 | MAP_RS06740 | Uncharacterized protein | Extracellular |
| MAP1405 | 41395856 | MAP_RS07135 | Channel-forming protein | Outer Membrane |
| MAP1476c | 41395927 | MAP_RS07510 | Cutinase | |
| MAP1507* | 41395958 | MAP_RS07675 | PE family protein | Periplasmic |
| MAP1509* | 41395960 | MAP_RS07690 | ESX secretion-associated | |
| | | | protein EspG | |
| MAP1530¹ | 41395981 | MAP_RS07800 | Hypothetical protein | Periplasmic |
| MAP1549c¹ | 41396000 | MAP_RS07885 | Malate synthase G | Moonlighting proteins |
| MAP1565 | 41396016 | MAP_RS07965 | Molybdate-binding protein | Periplasmic |
| MAP1588c¹ | 41396039 | MAP_RS08080 | Alkylhydroperoxidase | Moonlighting proteins |
| MAP1595 | 41396046 | MAP_RS08115 | Bacterioferritin | |
| MAP1607c* | 41396058 | MAP_RS08175 | Resuscitation-promoting | Non-cytoplasmic |
| | | | factor RpfC | (sigl sequence) |
| MAP1609c* | 41396060 | MAP_RS08185 | Esterase family protein | Extracellular |
| MAP1653 | 41396105 | MAP_RS08400 | 2-Cys peroxiredoxin | Periplasmic |
| MAP1680c | 41396132 | MAP_RS08540 | Cutinase family protein | |
| MAP1725c | 41396177 | MAP_RS08780 | Catalase | Periplasmic |
| MAP1769c | 41396221 | MAP_RS09000 | Sugar ABC transporter | Periplasmic |
| | | | substrate-binding protein | |
| MAP1781* | 41396233 | MAP_RS09060 | Hypothetical protein | |
| MAP1784c | 41396236 | MAP_RS09075 | Uncharacterized protein | Extracellular |
| MAP1858 | 41396310 | MAP_RS09440 | Mannan-binding protein | Periplasmic |
| MAP1928c | 41396381 | MAP_RS09795 | Endopeptidase | Periplasmic |
| MAP1962 | 41396415 | MAP_RS09970 | Type I glutamate-ammonia | |
| | | | ligase | |
| MAP1966c | 41396419 | MAP_RS09990 | Type I glutamate-ammonia | |
| | | | ligase | |
| MAP1997 | 41396450 | MAP_RS10150 | Acyl carrier protein | |
| MAP2057 | 41396510 | MAP_RS10450 | Haloalkane dehalogenase | Periplasmic |
| MAP2064 | 41396517 | MAP_RS10485 | Sensor domain-containing | Extracellular |
| | | | protein | |
| MAP2069c | 41396522 | MAP_RS10510 | Molecular chaperone HtpG | |
| MAP2109c | 41396562 | MAP_RS10720 | Uncharacterized protein | Outer Membrane |
| MAP2120c | 41399240 | MAP_RS10780 | Cysteine desulfurase | Moonlighting proteins |
| MAP2121c¹ | 41396574 | MAP_RS10785 | Hypothetical protein | |
| MAP2167c | 41396620 | MAP_RS11025 | Hypothetical protein | |
| MAP2168c | 41396621 | MAP_RS11030 | Hypothetical protein | |
| MAP2213c | 41396667 | MAP_RS11255 | Sulfate ABC transporter | Periplasmic |
| | | | substrate-binding protein | |
| MAP2216c | 41396670 | MAP_RS11270 | Sensor domain-containing | Periplasmic |
| | | | protein | |
| MAP2268c | 41396722 | MAP_RS11540 | Nucleoside-diphosphate | |
| | | | kinase | |
| MAP2273c* | 41396727 | MAP_RS11565 | Resuscitation-promoting | |
| | | | factor RpfE | |
| MAP2283 | 41396737 | MAP_RS11625 | Esterase | Periplasmic |
| MAP2286c | 41396740 | MAP_RS11640 | DBSA oxidoreductase | |
| MAP2498c | 41396954 | MAP_RS12745 | Uncharacterized protein | Extracellular |
| MAP2506c | 41396962 | MAP_RS12780 | Uncharacterized protein | Periplasmic |
| MAP2523c | 41396979 | MAP_RS12865 | Uncharacterized protein | Periplasmic |
| MAP2552 | 41397008 | MAP_RS13020 | Lytic transglycosylase | Extracellular |
| MAP2555c* | 41397011 | MAP_RS13035 | Serine protease | Periplasmic |
| MAP2576c* | 41397032 | MAP_RS13145 | Hypothetical protein | Periplasmic |
| MAP2600 | 41397056 | MAP_RS13265 | PPE family protein | Extracellular |
| MAP2625 | 41397081 | MAP_RS13390 | Uncharacterized protein | Extracellular |
| MAP2626 | 41397082 | MAP_RS13395 | Uncharacterized protein | Periplasmic |
| MAP2698c* | 41397155 | MAP_RS13745 | Acyl-ACP desaturase | |
| MAP2744c | 41397201 | MAP_RS13985 | Catalase-related peroxidase | Periplasmic |
| MAP2746 | 41397203 | MAP_RS13995 | Hypothetical protein | |
| MAP2832 | 41397289 | MAP_RS14485 | Alpha/beta hydrolase | Extracellular |
| MAP2855c* | 41397312 | MAP_RS14610 | Hypothetical protein | |
| MAP2979 | 41397436 | MAP_RS15250 | D-alanyl-D-alanine | Periplasmic |
| | | | carboxypeptidase | |
| MAP3007 | 41397925 | MAP_RS15390 | Aldo/keto reductase | |
| MAP3092 | 41398010 | MAP_RS15855 | Iron citrate ABC transporter | Periplasmic |
| | | | substrate-binding protein | |
| MAP3184¹ | 41398102 | MAP_RS16340 | PPE family protein | Extracellular |
| MAP3236 | 41398154 | MAP_RS16615 | Catalase HPII | |
| MAP3298 | 41398227 | MAP_RS16950 | NrdH-redoxin | Periplasmic |
| MAP3362 | 41398291 | MAP_RS17285 | Adenosylhomocysteinase | |
| MAP3384* | 41398313 | MAP_RS17400 | Copper-translocating P-type | |
| | | | ATPase | |
| MAP3387c* | 41398316 | MAP_RS17415 | Serine/threonine protein | Periplasmic |
| | | | kinase | |
| MAP3417c¹ | 41398346 | MAP_RS17565 | Esterase | Extracellular |
| MAP3456c | 41398385 | MAP_RS17770 | Isocitrate dehydrogenase, | |
| | | | NADP-dependent | |
| MAP3495c | 41398424 | MAP_RS17970 | Cutinase | |
| MAP3527 | 41398456 | MAP_RS18120 | Serine protease | Periplasmic |
| MAP3531c* | 41398460 | MAP_RS18140 | Esterase family protein | |
| MAP3550¹ | 41398480 | MAP_RS18240 | Alpha/beta hydrolase | Extracellular |
| MAP3613 | 41398543 | MAP_RS18550 | Uncharacterized protein | Extracellular |
| MAP3638 | 41398568 | MAP_RS18665 | Hemophore | Periplasmic |
| MAP3651c | 41398581 | MAP_RS18725 | Acyl-CoA dehydrogenase | |
| MAP3775c | 41398705 | MAP_RS19350 | ABC transporter | |
| MAP3785* | 41398715 | MAP_RS19400 | ESX secretion-associated | |
| | | | protein EspG | |
| MAP3802c | 41398733 | MAP_RS19495 | Membrane protein | Extracellular |
| MAP3836c¹ | 41398767 | MAP_RS19675 | Uncharacterized protein | Extracellular |
| MAP3840*¹ | 41398771 | MAP_RS19695 | Molecular chaperone DnaK | |
| MAP3875c | 41398806 | MAP_RS19880 | Uncharacterized protein | Periplasmic |
| MAP3902c | 41398833 | MAP_RS20015 | Uncharacterized protein | Extracellular |
| MAP3906 | 41398837 | MAP_RS20035 | Amidohydrolase | Extracellular |
| MAP3939c*¹ | 41398870 | MAP_RS20205 | PPE family protein | Cytoplasmic Membrane |
| MAP3951c | 41398882 | MAP_RS20265 | S9 family peptidase | Periplasmic |
| MAP3968*¹ | 41398899 | MAP_RS20350 | Hypothetical protein | |
| MAP3970 | 41398901 | MAP_RS20360 | Uncharacterized protein | Periplasmic |
| MAP3972c | 41398903 | MAP_RS20370 | Uncharacterized protein | Extracellular |
| MAP4034 | 41398965 | MAP_RS20680 | Uncharacterized protein | Periplasmic |
| MAP4142¹ | 41399074 | MAP_RS21255 | Elongation factor G | |
| MAP4143*¹ | 41399075 | MAP_RS21260 | Elongation factor Tu | |
| MAP4144 | 41399076 | MAP_RS21265 | PE family protein | Periplasmic |
| MAP4172c | 41399104 | MAP_RS21410 | Phosphate ABC transporter | Periplasmic |
| | | | substrate-binding protein PstS | |
| MAP4199* | 41399131 | MAP_RS21545 | Adenylate kinase | |
| MAP4236c | 41399168 | MAP_RS21730 | Cutinase family protein | |
| MAP4237c¹ | 41399169 | MAP_RS21735 | Cutinase | Extracellular |
| MAP4263¹ | 41399195 | MAP_RS21865 | tRNA (adenosine(37)-N6)- | Extracellular |
| | | | threonylcarbamoyltransferase | |
| | | | complex transferase subunit | |
| | | | TsaD | |
| MAP4265* | 41399197 | MAP_RS21875 | Molecular chaperone GroEL | |
| MAP4284* | 41399216 | MAP_RS21965 | Heavy metal translocating P- | |
| | | | type ATPase | |
| MAP4308c | 41396573 | MAP_RS22095 | Class I fructose-bisphosphate | Moonlighting proteins |
| | | | aldolase | |
| MAP4329c | 41399261 | MAP_RS22190 | Uncharacterized protein | Extracellular |
| MAP4339 | 41399271 | MAP_RS22255 | Thioredoxin-disulfide | |
| | | | reductase | |
| MAP4340* | 41399272 | MAP_RS22260 | Thiol reductase thioredoxin | |
| MAP4341 | 41399273 | MAP_RS22265 | N-acetymuramyl-L-alanine | |
| | | | amidase | |
| MAP4343c | 41399275 | MAP_RS22275 | Chromosome partitioning | |
| | | | protein ParB | |

*indicates MAP proteins with homology to other mycobacterial sp. proteins.
¹denotes proteins assayed for cell-mediated immune responses (Example 6, Table 4).

### Example 2

Identification of Putative M. bovis Antigens Using Reverse Vaccinology

Reverse vaccinology approaches, combining immunoinformatics, bacterial comparative genomics, and transcriptomics, were used to identify putative M. bovis diagnostic and vaccine antigens. Table 2 shows the putative M. bovis antigens identified from the strain AF2122-97 annotated genome (Malone K M et al. Genome Announcements (2017) 5 (14): e00157-17. doi: 10.1128/genomeA.00157-17.). Fourty-two of these antigens were proteins predicted to have an extracellular (13), periplasmic (27) or outer membrane localization (two) (see, Table 2).

TABLE 2
Putative M. bovis antigens. Their MAP orthologs are listed.

| M bovis | Protein | MAP | Subcellular |
|---|---|---|---|
| ID | Annotation | orthologs | Localization |
| Mb0485 | heparin binding hemagglutinin HBHA (adhesin) | MAP3968 | |
| Mb0869c | Oxidase | MAP0701c* | Periplasmic |
| Mb1397c | hypothetical protein | MAP0750c* | Outer Membrane |
| Mb1967 | thiol peroxidase | MAP1653* | Periplasmic |
| Mb2244 | glutamine synthetase GLNA1 (glutamine synthase) | MAP1962 | |
| | (GS-I) | | |
| Mb0064 | Oxidoreductase | MAP0081 | |
| Mb0303c | hypothetical protein | MAP2118 | |
| Mb1009 | serine protease | MAP0918* | Periplasmic |
| Mb2554c | hypothetical protein | MAP2334c | |
| Mb3065c | enoyl-CoA hydratase | MAP3087c | |
| Mb0192 | beta-glucosidase BGLS | MAP3625 | |
| Mb0341 | glucose-1-phosphate thymidylyltransferase | MAP3828 | |
| Mb1018c | putative serine rich protein | MAP0922c | |
| Mb1296 | hypothetical protein | MAP2506c* | Periplasmic |
| Mb2318 | haloalkane dehalogenase | MAP2057* | Periplasmic |
| Mb0290 | hypothetical protein | MAP3778 | |
| Mb0661c | methoxy mycolic acid synthase | MAP4116c | |
| Mb0975c | hypothetical protein | MAP0895c | |
| Mb1325 | diaminopimelate decarboxylase LysA | MAP2469c | |
| Mb3250c | RNA polymerase sigma factor RpoE | MAP3324c | |
| Mb1551 | Glycosyltransferase | MAP3762c | |
| Mb2215c | anthranilate phosphoribosyltransferase | MAP1931c | |
| Mb2270 | 3-oxoacyl-(acyl carrier protein) synthase II | MAP3485 | |
| Mb2472c | nucleoside diphosphate kinase | MAP2268c* | |
| Mb3631c | aspartate alpha-decarboxylase | MAP0457 | |
| Mb0041c | secreted proline rich protein MTC28 (proline rich 28 | MAP0047c | |
| | kDa antigen) | | |
| Mb0180 | MCE-family protein MCE1F | MAP3609 | |
| Mb3573c | acyl-CoA dehydrogenase FADE29 | MAP0524 | |
| Mb3761c | hypothetical protein | MAP0347c | |
| Mb3841 | hypothetical protein | MAP0209c | |
| Mb0250c | acyl-CoA dehydrogenase FADE5 | MAP3694c | |
| Mb1007 | two component response transcriptional regulatory | MAP0916 | |
| | protein MprA | | |
| Mb1950 | isocitrate lyase | MAP1643 | |
| Mb2258 | phosphotyrosine protein phosphatase PTPA | MAP1985 | |
| Mb1530 | methylmalonyl-CoA mutase | MAP1226 | |
| Mb2407c | salicylate synthase MbtI | MAP2205c | |
| Mb2953 | thioesterase TESA | MAP3745 | |
| Mb3314c | RNA polymerase sigma factor SigF | MAP3406c | |
| Mb3647 | lipoprotein LpqG | MAP0440c* | Periplasmic |
| Mb0130 | serine protease PepA | MAP3527* | Periplasmic |
| Mb0134c | secreted antigen 85-C (AG58C) (Mycolyl transferase | MAP3531c* | |
| | 85C) (fibronectin-binding protein C) | | |
| Mb0297 | hypothetical protein | MAP3785* | |
| Mb0476 | isocitrate lyase | MAP3961 | |
| Mb0479c | mycolic acid synthase PcaA | MAP3964c | |
| Mb0511 | pyrroline-5-carboxylate reductase | MAP3991 | |
| Mb0604 | MCE-family protein MCE2A | MAP4084 | |
| Mb0796c | bifunctional cephalosporin acylase/gamma- | MAP0607c | |
| | glutamyltranspeptidase | | |
| Mb0927c | two component response transcriptional regulatory | MAP0834c | |
| | protein PRRA | | |
| Mb1253 | RNA polymerase sigma factor SigE | MAP2557c | |
| Mb1381 | acyl-CoA dehydrogenase | MAP1553c | |
| Mb1446c | lipoprotein LprG | MAP1138c* | |
| Mb1788 | cutinase Cut1 | MAP1476c* | Extracellular |
| Mb1819 | PE family protein | MAP1507 | Periplasmic |
| Mb1822 | hypothetical protein | MAP1509* | |
| Mb1848 | hypothetical protein | MAP1530* | Periplasmic |
| Mb1852 | preprotein translocase subunit SecA | MAP1534 | |
| Mb1917c | chorismate mutase | MAP1608c | |
| Mb1918c | secreted antigen 85-B fbpB (Mycolyl transferase | MAP1609c* | |
| | 85B) (fibronectin-binding protein B) (extracellular | | |
| | alpha-antigen) | | |
| Mb1945c | hypothetical lipoprotein | MAP1638c | |
| Mb1946c | lipoprotein LppC | MAP1638c | |
| Mb2124c | hypothetical protein | MAP1833c | |
| Mb2406 | putative acetyl hydrolase MBTJ | MAP2197 | |
| Mb2422c | sulfate-binding lipoprotein | MAP2213c* | Periplasmic |
| Mb2490 | esterase/lipase LipP | MAP2283* | Periplasmic |
| Mb2614c | GTP pyrophosphokinase | MAP1047 | |
| Mb2722 | RNA polymerase sigma factor | MAP2820 | |
| Mb2734 | Hydrolase | MAP2832* | Extracellular |
| Mb2935 | D-alanyl-D-alanine carboxypeptidase | MAP2979* | Periplasmic |
| Mb3011c | isopropylmalate isomerase small subunit | MAP3025c | |
| Mb3116 | chain-fatty-acid-CoA ligase | MAP2874c | |
| Mb3157c | two component transcriptional regulatory protein | MAP3271c | |
| | DevR | | |
| Mb3175 | NADH dehydrogenase subunit G | MAP3207 | |
| Mb3450 | transcriptional regulatory protein WHIB-like WHIB3 | MAP4273c | |
| Mb3453c | putative DNA-binding/iron metalloprotein/AP | MAP4263* | Extracellular |
| | endonuclease | | |
| Mb3469c | hypothetical protein | MAP4249 | |
| Mb3481 | cutinase precursor CUT3 | MAP4237c* | Extracellular |
| Mb3526c | MCE-family protein MCE4D | MAP0567* | |
| Mb3751 | cutinase precursor CUT5 | MAP0333* | Extracellular |
| Mb3789 | 19 kDa lipoprotein antigen precursor LPQH | MAP0261c* | |
| Mb3833c | secreted MPT51/MPB51 antigen protein FBPD | MAP0217* | Extracellular |
| | (AG58C) (Mycolyl transferase 85C) (fibronectin- | | |
| | binding protein C) | | |
| Mb3834c | secreted antigen 85-A FBPA (Mycolyl transferase | MAP0216* | |
| | 85A) (fibronectin-binding protein A) (antigen 85 | | |
| | complex A) | | |
| Mb3840 | exported repetitive protein precursor PirG (cell | MAP0210c | |
| | surface protein) (EXP53) | | |
| Mb3876 | superoxide dismutase [Fe] SODA | MAP0187c* | Periplasmic |
| Mb3922c | PE family protein | MAP0157* | |
| Mb3927c | hypothetical protein | MAP4325c | |
| Mb0014c | transmembrane serine/threonine-protein kinase B | MAP0016c | |
| | PKNB (protein kinase B) (STPK B) | | |
| Mb0015c | transmembrane serine/threonine-protein kinase A | MAP0018c | |
| | PKNA (protein kinase A) (STPK A) | | |
| Mb0176 | MCE-family protein MCE1B | MAP3605 | |
| Mb0177 | MCE-family protein MCE1C | MAP3606 | |
| Mb0178 | MCE-family protein MCE1D | MAP3607 | |
| Mb0179 | MCE-family lipoprotein LprK | MAP3608 | |
| Mb0184 | mce associated membrane protein | MAP3613* | Extracellular |
| Mb0209 | hypothetical protein | MAP3638* | Periplasmic |
| Mb0291 | hypothetical protein | MAP3779 | |
| Mb0293 | PE family protein | MAP3781 | |
| Mb0295 | hypothetical protein | MAP3783 | |
| Mb0299 | protease precursor | MAP3787 | |
| Mb0317 | hypothetical protein | MAP3796 | |
| Mb0368 | hypothetical protein | MAP3863c | |
| Mb0418c | serine/threonine-protein kinase PKNG (protein | MAP3893c | |
| | kinase G) (STPK G) | | |
| Mb0419c | glutamine-binding lipoprotein | MAP3894c | |
| Mb0426 | lipoprotein aminopeptidase LpqL | MAP3906 | Extracellular |
| Mb0459c | membrane protein | MAP1241c | |
| Mb0466c | Peptidase | MAP3951c* | Periplasmic |
| Mb0487 | hypothetical protein | MAP3970* | Periplasmic |
| Mb0501 | two component sensory transduction protein RegX3 | MAP3983 | |
| Mb0515c | cyclopropane-fatty-acyl-phospholipid synthase 2 | MAP3995c | |
| Mb0605 | MCE-family protein MCE2B | MAP4085 | |
| Mb0606 | MCE-family protein MCE2C | MAP4086 | |
| Mb0607 | hypothetical protein | MAP4087 | |
| Mb0610 | MCE-family protein MCE2F | MAP4089 | |
| Mb0690 | lipoprotein LpqP | MAP4288 | |
| Mb0780 | two component system response transcriptional | MAP0591 | |
| | positive regulator PHOP | | |
| Mb0781 | two component system response sensor kinase | MAP0592* | |
| | membrane associated PHOR | | |
| Mb0803 | phosphoribosylaminoimidazole-succinocarboxamide | MAP0614 | |
| | synthase | | |
| Mb0804 | protease II PrtB | MAP0615* | Periplasmic |
| Mb0845c | hypothetical protein | MAP0656c | |
| Mb0858 | lipoprotein LpqQ | MAP1216c | |
| Mb0896c | PE-PGRS family protein PGRS15 | MAP4144* | Extracellular |
| Mb0994 | metal cation transporter P-type ATPase CtpV | MAP4284* | |
| Mb1036 | resuscitation-promoting factor rpfB | MAP0974* | Periplasmic |
| Mb1044c | putative lipoprotein LpqT | MAP0981c* | Extracellular |
| Mb1050 | putative lipoprotein LpqU | MAP0989 | |
| Mb1188c | hypothetical protein | MAP2626* | Periplasmic |
| Mb1195 | respiratory nitrate reductase subunit delta NarJ | MAP2618c | |
| Mb1217c | acyl-CoA synthetase | MAP2596 | |
| Mb1255 | serine protease HtrA | MAP2555c* | Periplasmic |
| Mb1262c | hypothetical protein | MAP2552* | Extracellular |
| Mb1297c | transmembrane serine/threonine-protein kinase H | MAP2504 | |
| Mb1311c | periplasmic oligopeptide-binding lipoprotein OppA | MAP2491 | |
| Mb1724 | hypothetical protein | MAP1405* | Outer Membrane |
| Mb1757c | hypothetical protein | MAP2475 | |
| Mb1775 | anchored-membrane serine/threonine-protein kinase | MAP1332 | |
| | PKNF (protein kinase F) (STPK F) | | |
| Mb1783c | hypothetical protein | MAP0847 | |
| Mb1816 | PE family protein | MAP1507 | Periplasmic |
| Mb1826 | hypothetical protein | MAP1513 | |
| Mb1888 | molybdate-binding lipoprotein | MAP1565* | Periplasmic |
| Mb1943c | catalase-peroxidase-peroxynitritase T KATG | MAP1668c | |
| Mb2006c | cutinase precursor CFP21 | MAP1680c* | Extracellular |
| Mb2164c | hypothetical protein | MAP1885c | |
| Mb2323 | cutinase CUT2 | MAP1662c | |
| Mb2401c | peptide synthetase MBTE (peptide synthase) | MAP2173c | |
| Mb2404c | phenyloxazoline synthase MBTB (phenyloxazoline | MAP2177c | |
| | synthetase) | | |
| Mb2405 | bifunctional salicyl-AMP ligase/salicyl-S-arcp | MAP2178 | |
| | synthetase | | |
| Mb2454 | alkyl hydroperoxide reductase subunit C | MAP1589c | |
| Mb2606c | hypothetical protein | MAP1056 | |
| Mb2613 | peptidyl-prolyl cis-trans isomerase B | MAP1050c* | Periplasmic |
| Mb2740c | hypothetical protein | MAP2837c | |
| Mb2878 | PE-PGRS family protein PGRS48 | MAP4144* | Extracellular |
| Mb2966 | acyl-CoA synthetase | MAP3752 | |
| Mb2975c | Oxidoreductase | MAP0059c | |
| Mb2976 | methyltransferase (methylase) | MAP3760c | |
| Mb3010c | DNA-binding protein HU | MAP3024c | |
| Mb3070 | FEIII-dicitrate-binding periplasmic lipoprotein | MAP3092* | Periplasmic |
| Mb3298 | metal cation-transporting P-type ATPase C CtpC | MAP3384* | |
| Mb3389 | hypothetical protein | MAP3461 | |
| Mb3476c | hypothetical protein | MAP4242 | |
| Mb3525c | MCE-family lipoprotein LprN | MAP0568 | |
| Mb3574c | acyl-CoA dehydrogenase FADE28 | MAP0523 | |
| Mb3618c | hypothetical protein | MAP0471 | Periplasmic |
| Mb3632c | pantoate-beta-alanine ligase | MAP0456 | |
| Mb3690c | periplasmic dipeptide-binding lipoprotein DppA | MAP0409* | Periplasmic |
| Mb3832c | hypothetical protein | MAP0218* | Periplasmic |
| Mb3850c | polyketide synthase associated protein PapA2 | MAP1694 | |
| Mb3918c | hypothetical protein | MAP0162* | |
| Mb3921c | PPE family protein | MAP0158 | |
| Mb3941 | RNA polymerase sigma factor SigM | MAP4337 | |
| Mb1214 | polyketide synthase associated protein PapA3 | MAP2231 | |
| Mb1267 | sugar-binding lipoprotein LpqY | MAP2548c | |
| Mb1379 | acyl carrier protein | MAP1555c | |
| Mb1382c | hypothetical protein | MAP3149c | |
| Mb1553c | Glycosyltransferase | MAP3762c | |
| Mb1811 | hypothetical protein | MAP1501 | |
| Mb1814 | putative ferredoxin | MAP1504 | |
| Mb1817 | PPE family protein | MAP1506 | |
| Mb1820 | hypothetical protein | MAP1508 | |
| Mb1825 | hypothetical protein | MAP1512 | |
| Mb2248c | exported protease | MAP1968c | |
| Mb2398c | putative protein MbtH | MAP1872c | |
| Mb2399c | lysine-N-oxygenase MBTG (L-lysine 6- | MAP2170c | |
| | monooxygenase) (lysine N6-hydroxylase) | | |
| Mb2400c | peptide synthetase MBTF (peptide synthase) | MAP2171c | |
| Mb2439c | hypothetical protein | MAP2325 | |
| Mb2980 | hypothetical protein | MAP1233 | |
| Mb3474c | hypothetical protein | MAP4244 | |
| Mb3475c | hypothetical protein | MAP4243 | |
| Mb3479 | secreted serine protease | MAP4239c | |
| Mb3524c | MCE-family protein MCE4F | MAP0569 | |
| Mb3528c | MCE-family protein MCE4B | MAP0565 | |
| Mb3529c | MCE-family protein MCE4A | MAP0564 | |
| Mb3879 | hypothetical protein | MAP0185c | |
| Mb3890 | hypothetical protein | MAP0171c | |
| Mb3915c | hypothetical protein | MAP0166 | |
| Mb3916c | secreted alanine and proline rich protease | MAP0165 | |
| Mb3919c | putative ESAT-6 like protein 11 | MAP0161 | |
| Mb3920c | hypothetical protein | MAP0160 | |
| Mb3925c | hypothetical protein | MAP4323c | |

*indicates that the MAP orthologs had been identified as a MAP antigen in Example 1 (Table 1).

Fifty-two of the MAP orthologs had been identified as MAP antigens by reverse vaccinology (Table 1). This important number of common antigens supports the parallel approach used for the identification of antigens for both MAP and M. bovis.

### Example 3

MAP and M. bovis Gene Synthesis, Cloning, and Recombinant Protein Expression

MAP and M. bovis genes were codon optimized in silico for protein expression in E. coli and synthesized as double-stranded DNA fragments (GeneArt™; ThermoFisher Scientific, Waltham, MA) for direct cloning into the Gateway expression vector pET301/CT-Dest (Invitrogen, Carlsbad, CA). Recombinant plasmids were transformed into E. coli BL21Star (DE3) competent cells (ThermoFisher Scientific, Waltham, MA) and verified by sequencing. E. coli cells containing recombinant plasmids were cultured in Lysogeny Broth supplemented with 100 μg/mL carbenicillin (Millipore Sigma, Burlington, MA) at 37° C. to an OD600 of 0.5-0.6. Recombinant protein expression was induced using 1 mM isopropyl β-d-1-thiogalactopyranoside (IPTG; Life Technologies, Carlsbad, CA) and further incubated for four hours at 37° C.

### Example 4

Affinity Purification of Polyhistidine-Tagged Recombinant MAP and M. bovis Proteins

Bacterial cell pellets were harvested by centrifugation and the pellets were suspended in lysis buffer (8 M urea, 500 mM NaCl, 100 mM NaH₂PO₄, 3-10 mM imidazole, and 10 mM Tris-HCl, pH 8) and homogenized by sonication. The homogenate was centrifuged for 10 minutes at 10,000×g and the clarified supernatant incubated with nickel-NTA agarose resin (Qiagen, Inc., Redwood City, CA) for 16-24 hours at 4° C. The resin was packed into a Poly-Prep chromatography column (Bio-Rad Laboratories, Inc., Hercules, CA) and washed with four bed volumes of lysis buffer followed by eight bed volumes of wash buffer (8 M urea, 500 mM NaCl, 100 mM NaH₂PO₄ and 10 mM Tris-HCl, pH 6.3). Polyhistidine-tagged recombinant MAP proteins were eluted from the nickel-NTA agarose resin by sequentially adding 1 bed volume of each buffer: Buffer D (8 M urea, 500 mM NaCl, 100 mM NaH₂PO₄, 8% glycerol and 10 mM Tris-HCl, pH 5.5), Buffer E (8 M urea, 500 mM NaCl, 100 mM NaH₂PO₄, 8% glycerol, and 10 mM Tris-HCl, pH 4.5), and 10 mM Tris-HCl, pH 8.0 containing 25 mM EDTA. Elution fractions were stored at −80° C. Protein integrity and purity were assessed by SDS-PAGE and amounts quantified using the Bio-Rad Protein Assay kit™ (Bio-Rad Laboratories, Inc., Hercules, CA).

### Example 5

### MAP Trial Design and MAP Infection of Calves

In each trial (1-7), 24 male Holstein calves were randomly assigned to one of four groups, each containing six calves. The first group of each trial received a sham vaccine consisting of adjuvant alone: 30% Emulsigen™ (Phibro Animal Health, Omaha, NE), 250 μg CpG ODN_2007 (BioSpring GmbH; Frankfurt, Germany) and phosphate-buffered saline (PBS). The other three groups were vaccinated with a unique pool of 5 recombinant MAP proteins (50 μg each) randomly selected from Table 1 and Table 6, formulated in the aforementioned adjuvant. Each animal trial consisted of: Primary immunization at Day 0 (4 weeks of age); Booster immunization at Day 28; Challenge with 3×10⁹ MAP CFUs in a surgically isolated intestinal segment at Day 56; and Euthanasia at Day 84. MAP challenge using surgically isolated intestinal segments was done essentially as described in Facciuolo et al., PLOS One (2016) 11: e0158747.

### Example 6

### Systemic Antibody Responses to Recombinant MAP Proteins

Antigen-specific IgG titres for all of the individual MAP proteins described in Table 1 and Table 6 were assayed in serum collected from the calves in Example 4 pre-vaccination (Day 0), one-month post-vaccination (Day 28), at the time of MAP challenge (Day 56) and at 28 days post-infection (Day 84). The analyst was blinded as to treatment group during the ELISA assays. Immulon™ 2 HB 96-well microtiter plates (ThermoFisher Scientific, Waltham MA) were coated with recombinant MAP protein (1 μg/mL) in bicarbonate-carbonate buffer, pH 9.5 overnight at 4° C. Plates were washed six times with water and blocked with Tris-buffered saline (TBS) supplemented with 1% fish gelatin (diluent) for 45 minutes at room temperature (RT). After washing twice with water and twice with diluent, four-fold serial dilutions of serum starting at 1 in 40 were added to duplicate wells and incubated for two hours at RT. Plates were subsequently washed six times with water and alkaline-phosphatase conjugated goat anti-bovine IgG (1 in 10,000 in diluent) added to each well and incubated for one hour at RT. Plates were washed as previously described and alkaline-phosphatase substrate PNPP (p-nitrophenyl phosphate; 1 mg/mL in PNPP buffer) added to each well and incubated for two hours at RT. Colorimetric reactions were stopped by adding 70 mM EDTA and absorbance measured at 405 and 490 nm (reference wavelength) using a SpectraMax Plus 384™ Reader (Molecular Devices, San Jose, CA). Antibody titres were determined using the reciprocal of the highest dilution that resulted in an absorbance value greater than the mean±2 standard deviations (SD) of the absorbance value from serum samples obtained from Day 0 calves. A Student's two-tailed t-test was used to compare IgG titres in MAP-infected, vaccinated calves to MAP-infected, unvaccinated calves. p values less than 0.05 were considered statistically significant.

### Example 7

### Systemic and Mucosal Cell-Mediated Responses to Recombinant MAP Proteins

PBMCs were isolated as described in Charavaryamath et al., Clin. Vaccine Immunol. (2013) 20:156-165. Mucosal leukocytes were isolated from MAP-infected surgically isolated intestinal segments as described in Facciuolo et al., PLOS One (2016) 11: e0158747. See Table 1 and Table 6 for the MAP proteins used in this Example.

PBMCs (5×10⁶) and mucosal leukocytes (2×10⁶) were seeded in 12-well tissue culture plates at final volume of 1 mL in complete medium (DMEM supplemented with 10% fetal bovine serum (FBS) plus antibiotics, antimycotics, and 10 μg/mL gentamicin). Cultures were stimulated with medium alone or 2.5 μg/mL recombinant MAP protein, prepared in complete medium, at 37° C. under 5% CO₂ in a humidified chamber. At 24 hours post-stimulation, cells in suspension were collected and centrifuged for seven minutes at 300×g, and 1 mL of TRIzol Reagent™ (Invitrogen, Carlsbad, CA) applied to each well to detach & lyse adherent cells. After centrifugation the supernatant was discarded and TRIzol Reagent™ (Invitrogen, Carlsbad, CA) from the corresponding well used to lyse the pelleted cells. Samples were subsequently incubated at RT for 10-15 minutes before storing at −80° C.

Cells lysed with TRIzol Reagent™ (Invitrogen, Carlsbad, CA) were extracted once with chloroform (0.2 mL/mL TRIzol Reagent™, Invitrogen, Carlsbad, CA) and RNA isolated from the aqueous phase using the RNeasy Mini Kit™ (Qiagen, Inc., Redwood City, CA) per the manufacturer's instructions. Samples were stored at −80° C. RNA integrity, quality and quantity were assessed using an Aglient 2100 BioAnalyzer and Nanodrop™ Spectrophotometer (ThermoFisher Scientific, Waltham, MA).

One μg of RNA was pre-treated to remove contaminating genomic DNA and reverse-transcribed using the QuantiTect Reverse Transcription Kit™ (Qiagen, Inc., Redwood City, CA) per the manufacturer's instructions. After cDNA synthesis, samples were diluted with RNase-, DNase-free water to a concentration of 5 ng/μL, and stored at −20° C. Real-time qPCR reactions were performed in triplicate with each reaction consisting of PerfeCTa SYBR Green SuperMix™ (QuantaBio, Beverly, MA), 300 nM of gene-specific primers and 25 ng of cDNA in a final volume of 15 μL. The thermal cycling program was two minutes at 95° C. for initial denaturation, followed by 36 cycles of 95° C. for 15 seconds, 60° C. for 30 seconds and 72° C. for 30 seconds, using a Bio-Rad CFX Connect Real-Time PCR Detection System™ (Bio-Rad Laboratories, Inc., Hercules, CA). Quantitative threshold cycle (Cq) for each reaction was determined by CFX Maestro™ Software (Bio-Rad Laboratories, Inc., Hercules, CA) and average Cq calculated using arithmetic average of triplicate reactions. Average Cq of each sample was normalized to the constitutively expressed gene YWHAZ (Puech et al., BMC Vet. Res. (2015) 11:65). Relative expression of IFNG (Osman et al., J. Gen. Virol. (2017) 98:1831-1842) and IL17A (Dadarwal et al., Theriogenology (2019) 126:128-139) was calculated using the equation 2-AACq as previously described in Pfaffl, M. W., Nuc. Acids Res. (2001) 29: e45). A Student's two-tailed t-test was used to compare responses in MAP-infected unvaccinated calves to MAP-infected vaccinates at 28 days post-infection. p values less than 0.1 were considered statistically significant.

Results of the experiments detailed in Examples 4-6 are shown in Tables 3 and 4. Table 3 shows the proteins tested from Table 1 and Table 6 that displayed statistically significant serum IgG antibody responses in MAP-challenged vaccinated calves when compared to MAP-challenged unvaccinated controls. Table 4 shows the proteins tested from Table 1 and Table 6 in which statistically significant antigen-specific cell-mediated responses were identified in isolated mucosal leukocytes from MAP-challenged vaccinated calves when compared to MAP-challenged unvaccinated controls. All the proteins listed in Tables 3 and 4 were identified by reverse vaccinology (Table 1), except MAP2785c in Table 3 and MAP1981c in Table 4.

TABLE 3
List of antigenic recombinant MAP proteins. Antibody
responses are represented as fold-change in serum IgG
titre in MAP-challenged vaccinates compared to MAP-
challenged non-vaccinates at 28 days post-infection.

| KEGG | NCBI | Trial/ | Antibody Response |
|---|---|---|---|
| Entry | Locus Tag | Group | (fold change) |
| MAP0157 | MAP_RS00790 | 1/B | 1454.4 |
| MAP0216 | MAP_RS01085 | 1/B | 464.3 |
| MAP0409 | MAP_RS02085 | 1/B | 550.5 |
| MAP0440c | MAP_RS02250 | 1/B | 452.9 |
| MAP3836c | MAP_RS19675 | 3/J | 642.7 |
| MAP0273 | MAP_RS01385 | 4/N | 4928.9 |
| MAP2855c | MAP_RS14610 | 4/N | 2272.4 |
| MAP3527 | MAP_RS18120 | 4/N | 2357.3 |
| MAP2785c | MAP_RS14245 | 4/O | 2322.7 |
| MAP2979 | MAP_RS15250 | 4/P | 1679.8 |
| MAP3092 | MAP_RS15855 | 4/P | 2030.1 |
| MAP0567 | MAP_RS02885 | 5/R | 679.1 |
| MAP0872 | MAP_RS04420 | 5/R | 726.1 |
| MAP0494 | MAP_RS02525 | 6/V | 736.1 |
| MAP0651 | MAP_RS03310 | 6/W | 640 |
| MAP0750c | MAP_RS03835 | 6/X | 1062.6 |
| MAP3184 | MAP_RS16340 | 6/X | 938.3 |
| MAP4339 | MAP_RS22255 | 7/Z | 1540.7 |
| MAP0145 | MAP_RS00735 | 7/Z | 1918 |
| MAP1595 | MAP_RS08115 | 7/Z | 2089.6 |
| MAP1962 | MAP_RS09970 | 7/Z | 682.9 |
| MAP2167c | MAP_RS11025 | 7/AA | 1666.7 |

TABLE 4
List of immunogenic recombinant MAP
proteins tested. In vitro re-stimulation assay to
test antigen-specific cell-mediated responses (IFNG and IL17A)
for individual recombinant MAP proteins in peripheral
blood mononuclear cells (PBMC) and mucosal leukocytes.

| | | | | | Mucosal | |
|---|---|---|---|---|---|---|
| KEGG | NCBI | Trial/ | PBMC | | leukocytes | |
| Entry | Locus Tag | Group | IFNG | IL17A | IFNG | IL17A |
| MAP3836c | MAP_RS19675 | 3/J | | + | | |
| MAP1981c | MAP_RS10065 | 3/J | + | + | | + |
| MAP4143 | MAP_RS21260 | 3/J | + | + | + | |
| MAP2121c | MAP_RS10785 | 3/J | | + | + | |
| MAP1588c | MAP_RS08080 | 3/K | + | + | + | + |
| MAP3840 | MAP_RS19695 | 3/K | | | + | |
| MAP4142 | MAP_RS21255 | 3/K | + | + | | + |
| MAP1530 | MAP_RS07800 | 3/K | | | | |
| MAP3968 | MAP_RS20350 | 3/K | | + | + | |
| MAP0144 | MAP_RS00730 | 3/L | + | | | |

(+) denotes vaccine-induced responses (p < 0.1) in MAP-challenged vaccinates compared to MAP-challenged non-vaccinates at 28 days post-infection.

### Example 8

M. bovis Trial Design and Infection of Mice

Four trials were conducted in mice to assess protection provided by immunization with pools of potential antigens against M. bovis challenge. In each trial (1-4), 40 female C57BL/6 mice of 6-7 weeks of age were randomly assigned to one of four groups, each containing ten animals. Administration of all vaccines was subcutaneous in a volume of 100 μL. The first group of each trial received a sham vaccine consisting of adjuvant alone: 30% Emulsigen™ (Phibro Animal Health), 10 μg CpG ODN_2007 (BioSpring GmbH; Frankfurt, Germany) and phosphate-buffered saline (PBS). In Trials 1 and 4, the second group of each trial received the BCG vaccine (Danish strain, 10⁶ CFUs). All the other groups were vaccinated with a unique pool of 5 recombinant M. bovis proteins (10 μg each) randomly selected from Table 2 and formulated in the aforementioned adjuvant.

Each mouse trial consisted of: Primary immunization at Day 0; Booster immunization at Day 30 except for the BCG group (no booster); Intranasal challenge with 10³ CFUs M. bovis at Day 56; and Euthanasia three weeks later or earlier if humane intervention for end of life was required.

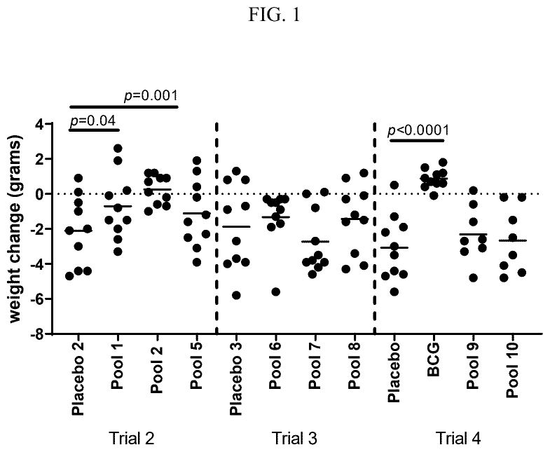

For Trials 2, 3 and 4, the weight of each mouse was recorded on the day of challenge and every day after that, until euthanasia. Statistical analysis (simple linear regression), was conducted on the weight values after controlling for cage effect. FIG. 1 shows the weight difference between the first day of record and the day of euthanasia, which occurred 17 days after challenge for Trial 2, 18 days after challenge for Trial 3 and 19 days after challenge for Trial 4. Mice immunized with a placebo control vaccine had lost a median weight of 2 g after 17-19 days post-challenge, while mice immunized with the BCG vaccine gained some weight (less than 1 g), which confirmed that the mouse model was appropriate to assess M. bovis infection. The antigen pools 1 and 2 induced a significantly lower weight loss than the placebo immunization, with p-values of 0.04 and 0.001 respectively. The p-value for the difference between placebo and antigen pool 5 immunizations is 0.14.

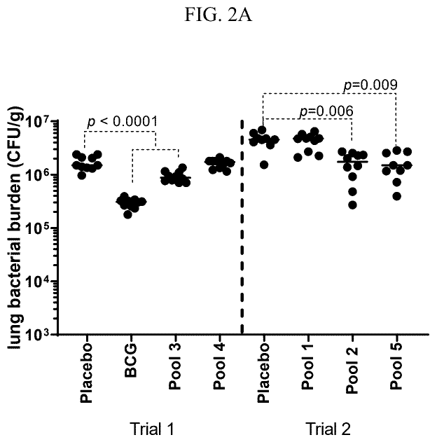

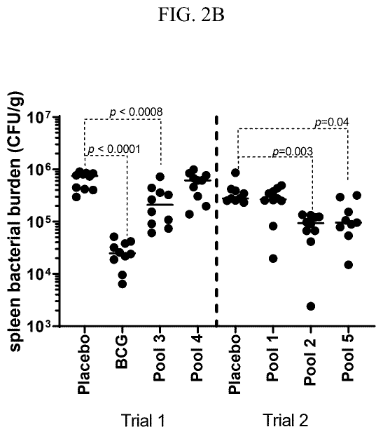

At euthanization, samples were collected from lung and spleen tissues to evaluate bacterial burden. Lung and spleen homogenates were prepared and plated on 7H11 agar plates; incubation at 37° C. was done over four weeks and the number of colonies per plate then counted. Results are expressed as mycobacterial CFU per gram of tissue and are interpreted relative to the CFU count of the placebo group. Results for Trials 3 and 4 show no difference between the placebo-immunized group and any of the antigen pool groups tested in these trials (pools 6, 7, 8, 9, and 10). FIGS. 2A and 2B show the results for Trials 1 and 2. As can be seen, placebo immunization induced significantly higher counts (simple linear regression, controlling for cage effects) of CFU in the lungs and spleens, respectively, than those induced by BCG administration and than those induced by antigen pools 2, 3, and 5. Antigen pools 2, 3, and 5 therefore protected the lungs and spleen of immunized animals from becoming abundantly infected by M. bovis.

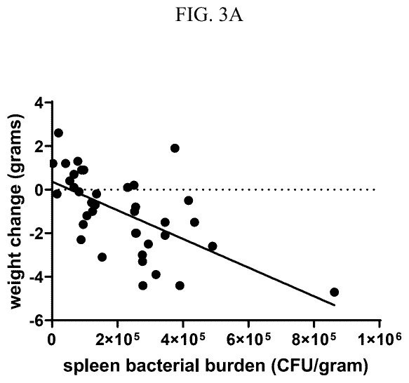

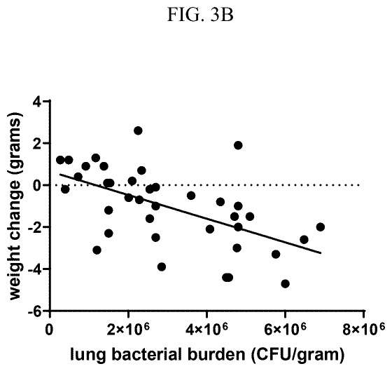

FIGS. 3A and 3B show the linear regression analysis of the weight gain and CFU counts, for lungs (FIG. 3A) and spleen (FIG. 3B), of the second mouse trial; all samples, whether from the BCG, placebo or test groups, were included. A weight loss is significantly associated with higher mycobacterial load; p-values are 0.0002 for lungs and <0.0001 for spleen. This further supports the use of the mouse model as an M. bovis screening tool.

### Example 9

Recall Assays with M. bovis Antigens

The ability of the 15 selected M. bovis antigens to stimulate the production of IFNγ by T cells isolated from M. bovis challenged animals was evaluated individually by recall assays. Six calves, labelled 74, 76, 77, 78, 79 and 81, were challenged by aerosol route with 1×10⁴ CFU/animal. At each of three time points (Day 0 of challenge, Day 28 and Day 42), whole blood was collected into lithium heparin tubes from every animal.

0.5 mL of blood was added to wells in multi-well plates containing a negative control (PBS), a positive control (bPPD with a final concentration of 300 IU/mL as per manufacturer's instructions) or one of the 15 individual proteins (final concentration of 5 μg/mL). The blood plus antigen or PBS were mixed well and incubated at 37° C., 5% CO₂ for 24 hours.

After 24 hours, the whole blood was centrifuged and resulting clear plasma was collected and filtered. The filtrate was transferred from biosafety level 3 to biosafety level 2 where the IFNγ ELISA analysis was done.

The analyst was blinded as to treatment group during the ELISA assays. Immulon™ 2 HB 96-well microtiter plates (ThermoFisher Scientific, Waltham MA) were coated overnight with mouse anti recombinant bovine interferon γ (rBoIFNγ monoclonal antibody 2-2-1A) diluted 1:8000 in coating buffer. Plates were washed 4× with tris-buffered saline (TBS) supplemented with 0.05% v/v Tween-20 (TBST). Samples were applied in 100 μL volumes and diluted 1:4 in diluent (TBST supplemented with 0.1% w/v pig gelatin (MilliporeSigma Canada Co.)). Standard rBoIFNγ was prediluted to 2 ng/mL in fetal bovinse serum (FBS). The standard was diluted to 1000 μg/mL and two fold dilutions were done. Plates were then incubated two hours at room temperature or overnight at 4° C. Plates were washed 4× with TBST. Then 100 μL of detection antibody (rabbit anti recombinant bovine IFNγ (92-132) diluted 1:5000 in diluent) was added to each well for 1 hour incubation at room temperature. Plates were washed 4× with TBST. One hundred uL of biotin-conjugated goat anti rabbit IgG (Zymed 62-6140 or Fisher Invitrogen 656140), diluted 1:10,000 in diluent, was added to each well for a one-hour incubation at room temperature. Plates were washed 4× with TBST; then streptavidin-conjugated alkaline phosphatase (Jackson ImmunoResearch Laboratories, Inc.) diluted 1:5,000 in diluent was added to each well for 1 hour at room temperature. Finally, plates were washed 4× with TBST; then 100 μL of PNPP substrate (1 mg/mL in PNPP buffer) was added to each well for 30 minutes at room temperature. The reaction was stopped by the addition of 30 μL of 0.3 M EDTA and the plates were read at 2405 nm, reference 2490 nm. The IFNγ concentration of the samples was determined from the standard curve.

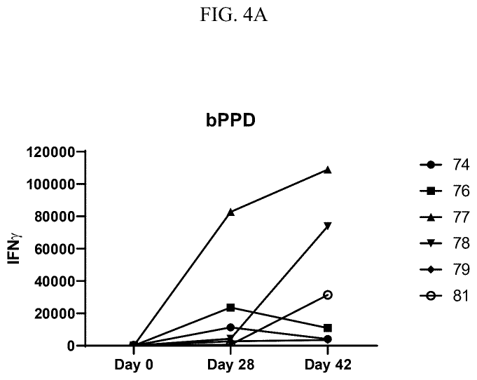

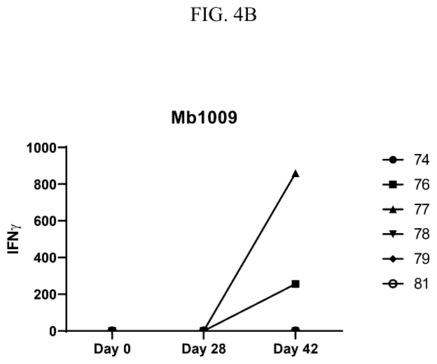

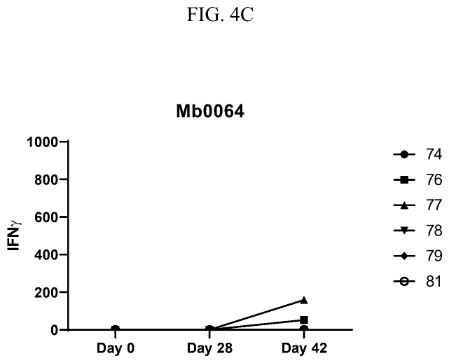

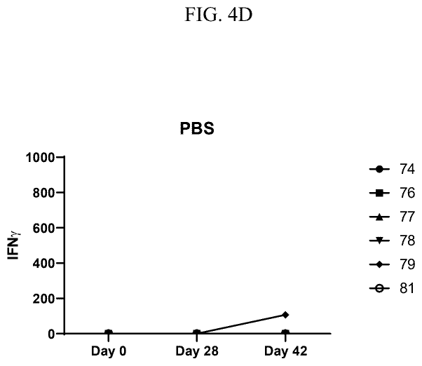

FIGS. 4A-4D show the IFNγ titres obtained by stimulation with the individual proteins that composed the three antigen pools (2, 3, and 5) showing some protection as assessed by CFU counts and weight gain. The splenocytes of calves numbered as 74, 76, 77, 78, 79 and 81, which were challenged with M. bovis, were stimulated with bPPD (positive control), PBS (negative control) or one of the 15 proteins as described above. bPPD (FIG. 4A) induced the release of IFNγ, however at very variable amounts among the animals. PBS (FIG. 4D) mostly did not induce any IFNγ release. Two M. bovis proteins, Mb 1009 (FIG. 4B) and Mb0064 (FIG. 4C) induced some light release of IFNγ.

### Example 10

Preselection of M. bovis Candidates

The 15 individual M. bovis proteins included in the antigen pools that showed protection in the mouse trials above are listed below with their MAP and Mtb orthologs. The identity between M. bovis and Mtb proteins is very high as expected and consistently above or equal to 99.7%.

The MAP orthologs of three of the 15 M. bovis proteins were also identified as potential antigens by reverse vaccinology; MAP2506c, MAP2057 and MAP0918 are the orthologs of Mb1296, Mb2318 and Mb 1009 respectively. Mb 1009 is one of the two M. bovis proteins that induced some IFNγ release in the recall assays.

TABLE 5
List of the individual M. bovis proteins included in the antigen pools that showed protection
in the mouse trials.

| | | | | | % homology | H37Rv | % identity |
|---|---|---|---|---|---|---|---|
| M. bovis | Protein | | Orthogroup | MAP | Mb/MAP | (Mtb) | Mb/Mtb |
| ID | Annotation | Localization | (TB Database) | orthologs | orthologs | orthologs | orthologs |
| Mb1018c | putative serine | Extracellular | 3774 | MAP0922c | 82.8 | Rv0991c | 100 |
| | rich protein | | | | | | |
| Mb0192 | beta-glucosidase | Periplasmic | 34660 | MAP3625 | 76.1 | Rv0186 | 99.9 |
| | BGLS | | | | | | |
| Mb1296 | hypothetical | Periplasmic | 3636 | MAP2506c | 84.9 | Rv1265 | 100 |
| | protein | | | | | | |
| Mb2318 | haloalkane | Periplasmic | 27399 | MAP2057 | 82.9 | Rv2296 | 100 |
| | dehalogenase | | | | | | |
| Mb0341 | glucose-1-phosphate | | 8835 | MAP3828 | 83.7 | Rv0034 | 100 |
| | thymidylyltransferase | | | | | | |
| Mb0064 | oxidoreductase | | 19919 | MAP0081 | 82.9 | Rv0063 | 100 |
| Mb0303c | hypothetical | | 2807 | MAP2118 | 74.5 | Rv0295c | 100 |
| | protein | | | | | | |
| Mb1009 | serine protease | Periplasmic | 33776 | MAP0918 | 72.8 | Rv0983 | 99.8 |
| Mb2554c | hypothetical | | 13983 | MAP2334c | 85.7 | Rv2525c | 100 |
| | protein, secreted; | | | | | | |
| | predicted to be a | | | | | | |
| | substrate of the | | | | | | |
| | twin arginine | | | | | | |
| | translocation (tat) | | | | | | |
| | export system | | | | | | |
| Mb3065c | enoyl-CoA | | 30706 | MAP3087c | 83 | Rv3039c | 100 |
| | hydratase | | | | | | |
| Mb3250c | RNA polymerase | | 34851 | MAP3324c | 93.9 | Rv3223c | 100 |
| | sigma factor (sigH) | | | | | | |
| Mb0290 | esx conserved | | 35494 | MAP3778 | 86.5 | Rv0282 | 99.8 |
| | component ecca3. | | | | | | |
| | esx-3 type vii | | | | | | |
| | secretion system | | | | | | |
| | protein | | | | | | |
| Mb1325 | diaminopimelate | | 33517 | MAP2469c | 85.7 | Rv1293 | 100 |
| | decarboxylase | | | | | | |
| Mb0661c | methoxy mycolic | | 25907 | MAP4116c | 88.9 | Rv0642c | 99.7 |
| | acid synthase 4 | | | | | | |
| Mb0975c | hypothetical | | 27069 | MAP0895c | 69.4 | Rv0950c | 100 |
| | protein | | | | | | |

Predicted MAP and Mtb orthologs are also listed.
If the MAP ortholog was identified as being a potential antigen by reverse vaccinology, its ID number is indicated in bold italics.
M. bovis proteins that induced IFNγ release in recall assays are indicated in bold.

Thus, immunogenic compositions and methods of making and using the same for controlling, preventing, and/or diagnosing mycobacterial infection, such as MAP and M. bovis infection using recombinant antigens are described. Although preferred embodiments of the subject invention have been described in some detail, it is understood that obvious variations can be made without departing from the spirit and the scope of the invention as defined by the appended claims.

1. An immunogenic, subunit composition comprising a pharmaceutically acceptable excipient and at least one isolated, mycobacterial antigen selected from (a) a Mycobacterium avium subspecies paratuberculosis (MAP) antigen, or an ortholog thereof, wherein the MAP antigen or ortholog is from Tables 1, 2, 3, 4, or 5; (b) a Mycobacterium bovis (M. bovis) antigen from Tables 2 or 5; an immunogenic fragment of (a) or (b); an immunogenic variant of (a) or (b); or the corresponding antigen from another mycobacterial strain or isolate, with the proviso that the selected mycobacterial antigen is not MAP 2785c or MAP 1981c.

2. The immunogenic composition of claim 1, wherein the MAP antigen is selected from one or more of the MAP antigens from Tables 3 or 4, or an immunogenic fragment or variant thereof.

3. The immunogenic composition of claim 1, wherein the MAP antigen or ortholog comprises an amino acid sequence with at least 99% sequence identity to a MAP antigen or ortholog from Tables 1, 2, 3, 4, or 5.

4. The immunogenic composition of claim 1, wherein the M. bovis antigen is selected from one or more of the M. bovis antigens from Table 5, or an immunogenic fragment or variant thereof.

5. The immunogenic composition of claim 1, wherein the M. bovis antigen comprises an amino acid sequence with at least 99% sequence identity to a M. bovis antigen from Tables 2 or 5.

6. The immunogenic composition of claim 1, wherein the mycobacterial antigen comprises a deletion of all or part of a transmembrane binding domain or a native signal sequence, if present.

7. The immunogenic composition of claim 1, comprising two or more isolated mycobacterial antigens, wherein the mycobacterial antigens are selected from MAP antigens or orthologs and/or M. bovis antigens or orthologs, from Tables 1, 2, 3, 4, 5, or 6, or an immunogenic fragment or variant thereof.

8. The immunogenic composition of claim 7, wherein the two or more antigens are provided as a fusion protein.

9. The immunogenic composition of claim 1, further comprising an immunological adjuvant.

10. The immunogenic composition of claim 9, wherein the immunological adjuvant comprises an oil-in-water emulsion.

11. The immunogenic composition of claim 9, wherein the immunological adjuvant comprises (a) a polyphosphazene; (b) a poly(I:C) or a CpG oligonucleotide; and (c) a host defense peptide.

12. The immunogenic composition of claim 11, wherein the immunological adjuvant is in the form of a microparticle.

13. A method of preventing and/or controlling a mycobacterial infection in a mammalian subject, wherein the mycobacterial infection is a MAP, M. bovis, or Mtb infection, the method comprising administering a therapeutic amount of the composition of claim 1 to the subject.

14. The method of claim 13, wherein the subject is a ruminant.

15. The method of claim 14, wherein the MAP infection comprises Johne's disease, or wherein the M. bovis or Mtb infection comprises tuberculosis.

16. The method of claim 13, wherein the subject is a human subject, and/or wherein the MAP infection comprises a gastrointestinal disorder, and/or wherein the M. bovis or Mtb infection comprises tuberculosis.

17. The method of claim 13, wherein preventing and/or controlling the mycobacterial infection in the mammalian subject comprises reducing colonization of the Mycobacterium in the mammalian subject.

18. The method of claim 13, wherein preventing and/or controlling the mycobacterial infection in the mammalian subject comprises reducing shedding of the Mycobacterium from the mammalian subject.

19. A method of detecting mycobacterial antibodies in a biological sample comprising:
(a) providing a biological sample;
(b) reacting said biological sample with one or more mycobacterial antigens from Tables 1, 2, 3, 4 or 5, an immunogenic fragment or variant thereof, or the corresponding antigen from another mycobacterial strain or isolate, under conditions which allow mycobacterial antibodies, when present in the biological sample, to bind to said one or more antigens to form an antibody/antigen complex; and
(c) detecting the presence or absence of said complex,
thereby detecting the presence or absence of mycobacterial antibodies in said sample.

20. An immunodiagnostic test kit for detecting mycobacterial infection according to the method of claim 19, said test kit comprising one or more mycobacterial antigens from Tables 1, 2, 3, 4 or 5, an immunogenic fragment or variant thereof, or the corresponding antigen from another mycobacterial strain or isolate, and instructions for conducting the immunodiagnostic test according to claim 19.
